(19) United States
(12) Patent Application Publication
(10) Pub. No.: US 2025/0000805 A1
(43) Pub. Date: Jan. 2, 2025

(54) VALBENAZINE COMPOSITIONS

(71) Applicant: Neurocrine Biosciences, Inc., San Diego, CA (US)

(72) Inventors: Richard Alexander Moore, Jr., San Diego, CA (US); Amelia M. Short, San Diego, CA (US); Zhonghui Huang, San Diego, CA (US)

(21) Appl. No.: 18/705,079

(22) PCT Filed: Oct. 28, 2022
(86) PCT No.: PCT/US2022/048181

§ 371 Date: Apr. 26, 2024

Related U.S. Application Data
(60) Provisional application No. 63/273,561, filed on Oct. 29, 2021.

(51) Int. Cl. A61K 9/48; A61K 9/16; A61K 31/4745

(52) U.S. Cl. CPC A61K 9/4866; A61K 9/1611; A61K 9/1652; A61K 9/1694; A61K 31/4745

(57) ABSTRACT

The present application relates to pharmaceutical dosage forms and solid dosage forms of L-Valine, (2R,3R,11bR)-1,3,4,6,7,11b-hexahydro-9,10-dimethoxy-3-(2-mehtylpropyl)-2H-benzo[a]quinolizin-2-yl ester (valbenazine) or a pharmaceutically acceptable salt thereof, including processes of preparation thereof, which are useful in the treatment of a neurological or psychiatric disease or disorder such as a hyperkinetic movement disorder.

### CROSS REFERENCE TO RELATED APPLICATIONS

This application claims the benefit under 35 U.S.C. § 119(e) of U.S. Provisional Application No. 63/273,561 filed on Oct. 29, 2021, which is incorporated herein by reference in its entirety.

### FIELD

This application relates to pharmaceutical compositions and solid dosage forms of L-Valine, (2R,3R,11bR)-1,3,4,6,7,11b-hexahydro-9,10-dimethoxy-3-(2-methylpropyl)-2H-benzo[a]quinolizin-2-yl ester (valbenazine, or “VBZ”) or a pharmaceutically acceptable salt thereof, including processes of preparation thereof, which are useful in the treatment of a neurological or psychiatric disease or disorder such as a hyperkinetic movement disorder.

### BACKGROUND

Dysregulation of dopaminergic systems is integral to several central nervous system (CNS) disorders, including neurological and psychiatric diseases and disorders. These neurological and psychiatric diseases and disorders include hyperkinetic movement disorders, and conditions such as schizophrenia and mood disorders. The transporter protein vesicular monoamine transporter-2 (VMAT2) plays an important role in presynaptic dopamine release and regulates monoamine uptake from the cytoplasm to the synaptic vesicle for storage and release.

L-valine, (2R3R,11bR)-1,3,4,6,7,11b-hexahydro-9,10-dimethoxy-3-(2-methylpropyl)-2H-benzo[a]quinolizin-2-yl ester, also known as valbenazine, is a selective VMAT2 inhibitor. A formulation of valbenazine:4-methylbenzenesulfonate (1:2), also referred to as valbenazine tosylate or valbenazine ditosylate, has been previously reported in the FDA approved drug label for INGREZZA®. It is manufactured in the form of size 1 hard gelatin capsules for a 40 mg unit dosage, as measured as the free base. Valbenazine readily hydrolyzes in the presence of heat and water. Partially due to its stability, attempts at formulating valbenazine have proven to be difficult, particularly for formulations suitable for oral administration to patients that experience difficulty swallowing tablets and capsules, such as patients with dysphagia, or pediatric patients.

Many patients experience difficulty swallowing tablets and capsules. This problem can lead to a variety of adverse events and patient noncompliance with treatment regimens. A survey of adults on difficulties swallowing tablets and capsules suggests that this problem may affect as many as 40 percent of Americans. Individuals who find it difficult to swallow tablets and capsules frequently cite the size as the main reason for the difficulty in swallowing.

Larger tablets and capsules have also been shown to affect the transit of drug products through the pharynx and esophagus. Larger tablets and capsules have been shown to have a prolonged esophageal transit time and may directly affect a patient's ability to swallow a particular drug product. This can lead to disintegration of the product in the esophagus and/or cause injury to the esophagus. The United States Food and Drug Administration (“FDA”) has indicated “that size should be considered as part of a single product risk/benefit profile.” The FDA further recommends “that the largest dimension of a tablet or capsule should not exceed 22 mm and that capsules should not exceed a standard 00 size.”

Accordingly, there is a need for improved pharmaceutical compositions of valbenazine, or a pharmaceutically acceptable salt thereof (e.g., valbenazine ditosylate), suitable for oral administration in the treatment of a neurological or psychiatric disease or disorder.

### SUMMARY

Provided is a pharmaceutical dosage form comprising a plurality of granules, wherein each granule comprises:
(a) an average diameter of at least 1 mm;
(b) at least one pharmaceutically acceptable carrier,
(c) an amount of valbenazine, or a pharmaceutically acceptable salt thereof; and
(d) a film-coating.

Also provided is a unit dosage form comprising the pharmaceutical dosage form, as described herein, wherein the valbenazine, or a pharmaceutically acceptable salt thereof, is present in an amount of about 10 mg to about 200 mg, based on the weight of the free base.

Also provided is a method of administering valbenazine, or a pharmaceutically acceptable salt thereof, to a patient in need thereof, comprising:
(a) providing the pharmaceutical dosage form or the unit dosage form, both as described herein;
(b) sprinkling the plurality of granules on soft food; and
(c) administering the soft food orally.

Also provided is a method of administering valbenazine, or a pharmaceutically acceptable salt thereof, to a patient in need thereof, comprising: orally administering the pharmaceutical dosage form or the unit dosage form, both as described herein.

Also provided is a method of administering valbenazine, or a pharmaceutically acceptable salt thereof, to a patient in need thereof, comprising: orally administering a capsule containing the pharmaceutical dosage form or the unit dosage, both as described herein.

Also provided is a method of treating a neurological or psychiatric disease or disorder in a patient in need thereof, comprising administering to the patient a therapeutically effective amount of the pharmaceutical dosage form or the unit dosage form, both as described herein. The present disclosure also provides a pharmaceutical dosage form disclosed herein for use in therapy. The present disclosure also provides a use of a pharmaceutical dosage form disclosed herein in the preparation of a medicament for treating a neurological or psychiatric disease or disorder.

These and other objects of the disclosure are described in the following paragraphs. These objects should not be deemed to narrow the scope of the disclosure.

### BRIEF DESCRIPTION OF THE DRAWINGS

FIG. 1. shows the oral dissolution profile of oral granules of valbenazine ditosylate.

FIG. 2. shows a comparison of dissolution profiles of the final blend powder, and the final blend powder compacted in a tablet press machine to obtain granules.

### DETAILED DESCRIPTION

This disclosure is directed to pharmaceutical dosage forms of valbenazine, or a pharmaceutically acceptable salt thereof (e.g., valbenazine ditosylate) having improved properties for oral administration. In particular, the pharmaceutical dosage forms can be prepared by processes involving elevated temperatures and aqueous environments, and can be easily orally administered to, for example, dysphagic patients and pediatric patients, using a vehicle (e.g., soft foods). The pharmaceutical dosage forms described herein can have a dissolution profile comparable to other dosage forms of valbenazine (e.g., a powder-filled capsule), but can have physical properties better suited to administration by sprinkling onto soft foods (e.g., uniform size, weight, and/or hardness). The pharmaceutical dosage forms comprise a plurality of granules, wherein each granule comprises a water-soluble film-coating, which can further improve the organoleptic properties of the sprinkled pharmaceutical composition.

A known dosage form of valbenazine comprises powder filled capsules. Due to the lubricant quantity in the powder filled capsule formulation, the blended powder therein can have a propensity for over-lubrication due to shear. Therefore, the powder blend filled in the capsule may be subject to variability in release (dissolution rate) when not compacted into a slug via a dosing disk/dosator. Because a product intended for “sprinkle” is removed from the capsule shell and broken up for ease of swallowing, the variability arising from shear impact may be uncontrolled if the powder is removed from the capsule shell.

By contrast, the granules described herein are prepared by compressing a final powder blend in an automated tablet press, thereby removing the variability associated with shear. The compression of the disintegrant components results in consistent and rapid dissolution, even in the sprinkle format.

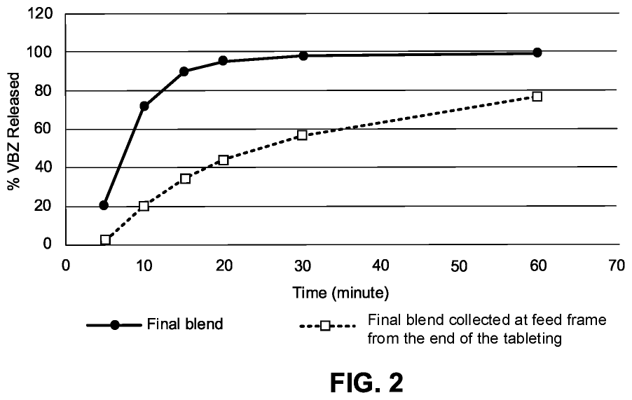

FIG. 2 shows that the granules described herein have a slower rate of dissolution compared to the powder.

In a fasted state, the Cₘₐₓ for valbenazine, when administered as granules described herein, sprinkled on applesauce, was decreased by about 28% when compared to the valbenazine commercial powder filled capsule (reference). This observation is consistent with a slightly reduced rate of absorption but similar extent of absorption. Advantageously, the delay in rate of absorption of valbenazine and associated reduced Cₘₐₓ did not affect the overall pharmacokinetics of valbenazine. Further, the oral granules described herein allow enough time for disintegration so as to pass through a gastronomy tube without clogging the tube.

### Compositions and Pharmaceutical Dosage Forms

Provided are pharmaceutical compositions of valbenazine and pharmaceutically acceptable salts thereof. The free base form of valbenazine has the formula:

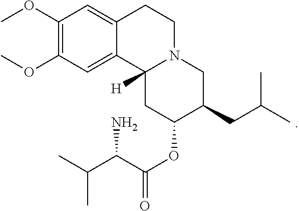 [SMILES: COc1cc2c(cc1OC)[C@H]1C[C@@H](OC(=O)[C@@H](N)C(C)C)[C@H](CC(C)C)CN1CC2]

Valbenazine can also be referred to as [(2R,3R,11bR)-9,10-dimethoxy-3-(2-methylpropyl)-2,3,4,6,7,11b-hexahydro-1H-benzo[a]quinolizin-2-yl] (2S)-2-amino-3-methylbutanoate, (S)-2-amino-3-methyl-butyric acid (2R,3R, 11bR)-3-isobutyl-9, 10-dimethoxy-1,3,4,6,7,11b-hexahydro-2H-pyrido[2,1-a]isoquinolin-2-yl ester, or NBI-98854. Valbenazine can also be referred to as L-Valine, (2R,3R,11bR)-1,3,4,6,7,11b-hexahydro-9,10-dimethoxy-3-(2-methylpropyl)-2H-benzo[a]quinolizin-2-yl ester. Certain active metabolites/degradation products of valbenazine are shown below:

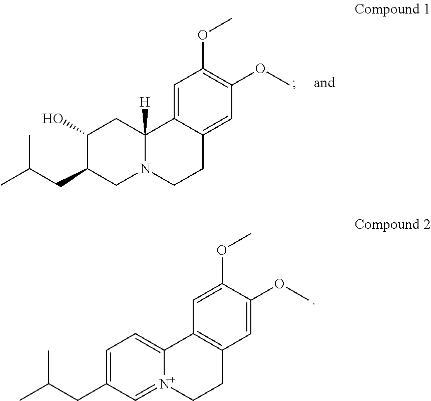

Valbenazine ditosylate refers to the 4-methylbenzenesulfonic acid salt of valbenazine, which is also known as INGREZZA®, valbenazine tosylate, valbenazine:4-methylbenzenesulfonate (1:2), or L-Valine, (2R,3R,11bR)-1,3,4,6,7,11b-hexahydro-9,10-dimethoxy-3-(2-methylpropyl)-2H-benzo[a]quinolizin-2-yl ester, 4-methylbenzenesulfonate (1:2).

Provided is a pharmaceutical dosage form comprising a plurality of granules, wherein each granule comprises:
(a) at least one pharmaceutically acceptable carrier;
(b) an amount of valbenazine, or a pharmaceutically acceptable salt thereof (e.g., valbenazine ditosylate); and
(c) a film-coating.

Provided is a pharmaceutical dosage form comprising a plurality of granules, wherein each granule comprises:
(a) an average diameter of at least 1 mm;
(b) at least one pharmaceutically acceptable carrier; and
(c) an amount of valbenazine, or a pharmaceutically acceptable salt thereof (e.g., valbenazine ditosylate).

Provided is a pharmaceutical dosage form comprising a plurality of granules, wherein each granule comprises:
(a) an average diameter of at least 1 mm;
(b) at least one pharmaceutically acceptable carrier,
(c) an amount of valbenazine, or a pharmaceutically acceptable salt thereof (e.g., valbenazine ditosylate); and
(d) optionally, a film-coating.

Provided is a pharmaceutical dosage form comprising a plurality of granules, wherein each granule comprises:
(a) an average diameter of at least 1 mm;
(b) at least one pharmaceutically acceptable carrier;
(c) an amount of valbenazine, or a pharmaceutically acceptable salt thereof; and
(d) a film-coating.

In some embodiments, each granule comprises a film-coating.

In some embodiments, each granule has an average diameter of about 1.5 mm to about 5 mm. In some embodiments, each granule has an average diameter of about 2 mm to about 3 mm. In some embodiments, each granule has an average diameter of about 2.15 mm to about 2.25 mm. In some embodiments, each granule has an average diameter of about 2.18 mm to about 2.23 mm. In some embodiments, each granule has an average diameter of about 2.19 mm to about 2.21 mm. In some embodiments, each granule has an average diameter of about 2.2 mm. In some embodiments, each granule has an average diameter of about 2 mm.

In some embodiments, each granule has an average diameter of about 1 mm to about 5 mm. In some embodiments, each granule has an average diameter of about 1 mm to about 4 mm. In some embodiments, each granule has an average diameter of about 1 mm to about 3.5 mm. In some embodiments, each granule has an average diameter of about 1.2 mm to about 5 mm. In some embodiments, each granule has an average diameter of about 1.2 mm to about 4 mm. In some embodiments, each granule has an average diameter of about 1.2 mm to about 3.5 mm.

In some embodiments, each granule has an average diameter of about 1.2 mm, about 1.4 mm, about 1.6 mm, about 1.8 mm, about 1.9 mm, about 2 mm, about 2.1 mm, about 2.2 mm, about 2.3 mm, about 2.4 mm, about 2.5 mm, about 2.6 mm, about 2.8 mm, or about 3 mm.

In some embodiments, each granule has a diameter variation of no more than 20% from the average diameter. In some embodiments, each granule has a diameter variation of no more than 15%, no more than 10%, no more than 7.5%, or no more than 5% from the average diameter. In some embodiments, each granule has a d99 particle diameter distribution of at most about 2.8 mm. In some embodiments, each granule has a d99 particle diameter distribution of at most about 2.5 mm. In some embodiments, each granule has a d99 particle diameter distribution of about 1.2 to about 2.8 mm, about 1.2 mm to about 2.5 mm, about 1.6 mm to about 2.8 mm, about 1.6 mm to about 2.5 mm, about 1.8 mm to about 2.8 mm, or about 1.8 mm to about 2.5 mm.

In some embodiments, each granule has an average density of at least about 0.5 g/cm³. In some embodiments, each granule has an average density of at least about 0.75 g/cm³, at least about 1 g/cm³, at least about 1.25 g/cm³, or at least about 1.5 g/cm³. In some embodiments, each granule has an average density of about 0.5 g/cm³ to about 2.5 g/cm³. In some embodiments, each granule has an average density of about 0.5 g/cm³ to about 2 g/cm³, about 0.75 g/cm³ to about 2.5 g/cm³, about 0.75 g/cm³ to about 2 g/cm³, about 1 g/cm³ to about 2.5 g/cm³, or about 1 g/cm³ to about 2 g/cm³. In some embodiments, each granule has an average density of about 1 g/cm³, about 1.25 g/cm³, about 1.5 g/cm³, about 1.75 g/cm³, about 2 g/cm³, or about 2.25 g/cm³, or about 2.5 g/cm³.

In some embodiments, each granule has an average diameter of about 1.2 mm to about 4 mm, an average diameter variation of no more than 1⁰% from the average diameter, and an average density of about 0.5 g/cm³ to about 2.5 g/cm³. In some embodiments, each granule has an average diameter of about 1.2 mm to about 4 mm, an average diameter variation of no more than 10% from the average diameter, and an average density of about 0.75 g/cm³ to about 2.5 g/cm³. In some embodiments, each granule has an average diameter of about 1.2 mm to about 3.5 mm, an average diameter variation of no more than 1⁰% from the average diameter, and an average density of about 0.75 g/cm³ to about 2.5 g/cm³. In some embodiments, each granule has an average diameter of about 1.2 mm to about 3.5 mm, an average diameter variation of no more than 10% from the average diameter, and an average density of about 1 g/cm³ to about 2 g/cm³.

In some embodiments, each granule has an average diameter of at most about 2.5 mm, a d99 particle diameter distribution of at most about 2.8 mm, and an average density of about 0.75 g/cm³ to about 2.5 g/cm³. In some embodiments, each granule has an average diameter of at most about 2.5 mm, a d99 particle diameter distribution of at most about 2.8 mm, and an average density of about 1 g/cm³ to about 2.5 g/cm³. In some embodiments, each granule has an average diameter of at most about 2.2 mm, a d99 particle diameter distribution of at most about 2.5 mm, and an average density of about 0.75 g/cm³ to about 2 g/cm³. In some embodiments, each granule has an average diameter of at most about 2.2 mm, a d99 particle diameter distribution of at most about 2.5 mm, and an average density of about 1 g/cm³ to about 2 g/cm³.

In some embodiments, each granule has an average hardness of about 0.5 kp to about 3 kp. In some embodiments, each granule has an average hardness of about 0.8 kp to about 2.6 kp. In some embodiments, each granule has an average hardness of about 1 kp to about 2.4 kp. In some embodiments, each granule has an average hardness of about 1.2 kp to about 2 kp. In some embodiments, each granule has an average hardness of about 1.4 kp to about 1.8 kp. In some embodiments, each granule has an average hardness of about 1.5 kp to about 1.7 kp. In some embodiments, each granule has an average hardness of about 1.6 kp.

In some embodiments, each granule has an average hardness of about 1 kp to about 2.5 kp. In some embodiments, each granule has an average hardness of about 1 kp to about 2 kp, about 1.2 kp to about 2.5 kp, or about 1.2 kp to about 2 kp. In some embodiments, each granule has an average hardness of about 1 kp, about 1.1 kp, about 1.2 kp, about 1.3 kp, about 1.4 kp, about 1.5 kp, about 1.6 kp, about 1.7 kp, about 1.8 kp, about 1.9 kp, or about 2 kp.

In some embodiments, each granule has an average weight of about 8 mg to about 11 mg. In some embodiments, each granule has an average weight of about 8 mg to about 10.2 mg. In some embodiments, each granule has an average weight of about 8 mg to about 10 mg. In some embodiments, each granule has an average weight of about 8.2 mg to about 10.1 mg. In some embodiments, each granule has an average weight of about 8.3 mg to about 9.9 mg. In some embodiments, each granule has an average weight of about 8.5 mg to about 9.7 mg. In some embodiments, each granule has an average weight of about 8.7 mg to about 9.5 mg. In some embodiments, each granule has an average weight of about 8.9 mg to about 9.3 mg. In some embodiments, each granule has an average weight of about 9 mg to about 9.2 mg. In some embodiments, each granule has an average weight of about 9.1 mg.

In some embodiments, each granule is stable to a film-coating process. In some embodiments, each granule is stable to a film-coating process at an elevated temperature (e.g., greater than 40° C.) in the presence of water. Each granule is stable to a film-coating process that can undergo a film-coating process at elevated temperature in the presence of water without resulting in significant degradation (e.g., via hydrolysis) of valbenazine or a pharmaceutically acceptable salt thereof. In some embodiments, less than about 10%, less than about 7.5%, less than about 5%, less than about 2.5%, or less than about 1% by weight of the valbenazine, or a pharmaceutically acceptable salt thereof, is degraded by the film-coating process. Film-coating processes which each granule is stable to include, for example, the Wurster technique, which can include the use of elevated temperatures (e.g., greater than 40° C.) in the presence of water.

In some embodiments, each granule comprises a film-coating. The film-coating described herein can comprise a film-forming polymer. In some embodiments, the film-coating comprises a film-forming polymer. As used herein, the term “film-forming polymer” refers to a polymer that can be applied (e.g., as a component of a film-coating) to the surface of each granule as described herein using aqueous coating techniques (e.g., Wurster coating) to provide a pharmaceutically acceptable film-coating over each granule. Exemplary film-forming polymers include, but are not limited to, hydroxypropyl methylcellulose (also referred to as hypromellose), sodium carboxymethyl cellulose, poly(vinyl alcohol), and methacrylic acid copolymer. In some embodiments, the film-forming polymer is poly(vinyl alcohol).

In some embodiments, the film-coating comprises a film-forming polymer, and the film-forming polymer is about 25 wt % to about 55 wt % of the weight of the film-coating. In some embodiments, the film-coating comprises a film-forming polymer, and the film-forming polymer is about 25 wt % to about 45 wt % of the weight of the film-coating. In some embodiments, the film-coating comprises a film-forming polymer, and the film-forming polymer is about 35 wt % to about 55 wt % of the weight of the film-coating. In some embodiments, the film-coating comprises a film-forming polymer, and the film-forming polymer is about 35 wt % to about 45 wt % of the weight of the film-coating. In some embodiments, the film-coating comprises a film-forming polymer, and the film-forming polymer is about 30 wt %, about 35 wt %, about 40 wt %, about 45 wt %, or about 50 wt % of the weight of the film-coating. In some embodiments, the film-forming polymer is poly(vinyl alcohol).

In some embodiments, the film-coating comprises a plasticizer. As used herein, the term “plasticizer” refers to a compound that can be added to a film-coating to decrease the glass-transition temperature and minimum film-forming temperature of the film-forming polymer. Exemplary plasticizers include, but are not limited to, polyethylene glycol, glycerin, triethyl citrate, and diethyl phthalate. In some embodiments, the plasticizer is polyethylene glycol, glycerin, or a mixture thereof. In some embodiments, the plasticizer is polyethylene glycol. In some embodiments, the plasticizer is polyethylene glycol (e.g., having a molecular weight of about 1,000 g/mol to about 4,000 g/mol).

In some embodiments, the film-coating comprises a plasticizer, and the plasticizer is about 5 wt % to about 30 wt % of the weight of the film-coating. In some embodiments, the film-coating comprises a plasticizer, and the plasticizer is about 5 wt % to about 25 wt %, about 10 wt % to about 30 wt %, or about 10 wt % to about 25 wt % of the weight of the film-coating. In some embodiments, the film-coating comprises a plasticizer, and the plasticizer is about 10 wt %, about 15 wt %, about 20 wt %, or about 25 wt % of the weight of the film-coating. In some embodiments, the plasticizer is polyethylene glycol.

The film-coating can further comprise a filler. In some embodiments, the film-coating comprises a filler. As used herein, the term “filler” refers to a compound that can be added to a film-coating to reduce the amount of other components in the composition. Certain fillers can improve the flowability of the film-coating. Exemplary fillers include, but are not limited to, talc, fumed silica, bentonite, and edible hydrogenated vegetable oils. In some embodiments, the filler is talc.

In some embodiments, the film-coating comprises a filler, and the filler is about 5 wt % to about 45 wt % of the weight of the film-coating. In some embodiments, the film-coating comprises a filler, and the filler is about 5 wt % to about 20 wt %, about 10 wt % to about 45 wt %, or about 10 wt % to about 20 wt % of the weight of the film-coating. In some embodiments, the film-coating comprises a filler, and the filler is about 10 wt %, about 15 wt %, about 20 wt %, about 25 wt %, about 30 wt %, about 35 wt %, or about 40 wt % of the weight of the film-coating. In some embodiments, the filler is talc.

The film-coating can further comprise a pigment/opacifier. In some embodiments, the film-coating comprises a pigment/opacifier. As used herein, the term “pigment/opacifier” refers to a compound that can be added to a film-coating to increase the opacity and/or provide color to coatings formed therefrom. Exemplary pigment/opacifiers include, but are not limited to, aluminum flakes, iron oxides, titanium dioxide, and natural colors. In some embodiments, the pigment/opacifier is titanium dioxide.

In some embodiments, the film-coating comprises a pigment/opacifier, and the pigment/opacifier is up to about 40 wt % of the weight of the film-coating. In some embodiments, the film-coating comprises a pigment/opacifier, and the pigment/opacifier is about 5 wt % to about 40 wt %, about 10 wt % to about 40 wt %, 20 wt % to about 40 wt %, or about 20 wt % to about 30 wt % of the weight of the film-coating. In some embodiments, the film-coating comprises a pigment/opacifier, and the pigment/opacifier is about 5 wt %, about 10 wt %, about 15 wt %, about 20 wt %, about 25 wt %, about 30 wt %, or about 35 wt % of the weight of the film-coating. In some embodiments, the pigment/opacifier is titanium dioxide.

In some embodiments, the film-coating comprises: (a) about 25 wt % to about 55 wt % of a film-forming polymer; (b) about 5 wt % to about 30 wt % of a plasticizer; (c) about 5 wt % to about 45 wt % of a filler; and (d) up to about 40 wt % of a pigment/opacifier; based on the weight of the film-coating. In some embodiments, the film-coating comprises: (a) about 25 wt % to about 55 wt % of poly(vinyl alcohol); (b) about 5 wt % to about 30 wt % of polyethylene glycol; (c) about 5 wt % to about 45 wt % of talc; and (d) up to about 40 wt % of titanium dioxide; based on the weight of the film-coating.

In some embodiments, the film-coating comprises a pH independent, water soluble polymeric film-coating system, comprising polymer, plasticizer, and optionally pigment, such as OPADRY® II. In some embodiments, the film-coating consists essentially of OPADRY® II. In some embodiments, the film-coating is OPADRY® II.

In some embodiments, the film-coating makes up about 3.5 wt % to about 15 wt % of the weight of each granule. In some embodiments, the film-coating makes up about 3.5 wt % to about 12.5 wt %, about 5 wt % to about 15 wt %, or about 5 wt % to about 12.5 wt % of the weight of each granule. In some embodiments, the film-coating makes up about 5 wt %, about 7.5 wt %, about 10 wt %, or about 12.5 wt % of the weight of each granule.

In some embodiments, the at least one pharmaceutically acceptable carrier comprises a diluent. As used herein, the term “diluent” refers to a compound that can dilute a composition. A diluent can also be referred to as a filler, dilutant or thinner. Exemplary diluents include, but are not limited to, lactose, lactose monohydrate, spray-dried monohydrate lactose, lactose-316 Fast Flo®, mannitol, isomalt, sucrose, dextrose, sorbitol, microcrystalline cellulose, silicified microcrystalline cellulose, acidified cellulose, starch 1500, prosolve MCC, colloidal silica, dicalcium phosphate dihydrate, and calcium carbonate, or any mixture thereof.

In some embodiments, the diluent is silicified microcrystalline cellulose, isomalt, or a mixture thereof. In some embodiments, the diluent is a mixture of silicified microcrystalline cellulose and isomalt.

In some embodiments, each granule comprises about 25 wt % to about 65 wt % of the diluent. In some embodiments, each granule comprises about 30 wt % to about 60 wt % of the diluent. In some embodiments, each granule comprises about 35 wt % to about 55 wt % of the diluent. In some embodiments, each granule comprises about 40 wt % to about 50 wt % of the diluent. In some embodiments, each granule comprises about 45 wt % of the diluent (e.g., a mixture of silicified microcrystalline cellulose and isomalt).

In some embodiments, each granule comprises about 25 wt %, about 30 wt %, about 35 wt %, about 40 wt %, about 45 wt %, about 50 wt %, about 55 wt %, or about 60 wt % of the diluent (e.g., a mixture of silicified microcrystalline cellulose and isomalt).

In some embodiments, each granule comprises a mixture of about 15 wt % to about 40 wt % of silicified microcrystalline cellulose and about 10 wt % to about 35 wt % isomalt as diluent. In some embodiments, each granule comprises a mixture of about 20 wt % to about 35 wt % of silicified microcrystalline cellulose and about 10 wt % to about 35 wt % isomalt as diluent. In some embodiments, each granule comprises a mixture of about 20 wt % to about 30 wt % of silicified microcrystalline cellulose and about 10 wt % to about 35 wt % isomalt as diluent.

In some embodiments, each granule comprises a mixture of about 15 wt % to about 40 wt % of silicified microcrystalline cellulose and about 15 wt % to about 30 wt % isomalt as diluent. In some embodiments, each granule comprises a mixture of about 20 wt % to about 35 wt % of silicified microcrystalline cellulose and about 15 wt % to about 30 wt % isomalt as diluent. In some embodiments, each granule comprises a mixture of about 20 wt % to about 30 wt % of silicified microcrystalline cellulose and about 15 wt % to about 30 wt % isomalt as diluent.

In some embodiments, each granule comprises a mixture of about 15 wt % to about 40 wt % of silicified microcrystalline cellulose and about 15 wt % to about 25 wt % isomalt as diluent. In some embodiments, each granule comprises a mixture of about 20 wt % to about 35 wt % of silicified microcrystalline cellulose and about 15 wt % to about 25 wt % isomalt as diluent. In some embodiments, each granule comprises a mixture of about 20 wt % to about 30 wt % of silicified microcrystalline cellulose and about 15 wt % to about 25 wt % isomalt as diluent.

In some embodiments, the at least one pharmaceutically acceptable carrier comprises a disintegrant. As used herein, the term “disintegrant” refers to a compound that can cause a composition to disintegrate and release its drug substance, e.g., on contact with moisture. Disintegrants can facilitate, e.g., the dosage form, to break up after oral administration. Exemplary disintegrants include, but are not limited to, croscarmellose sodium, crospovidone, starch (e.g., partially pregeletanized maize starch), cellulose, low substituted hydroxypropyl cellulose, alginic acid, sodium starch glycolate, and acid-carbonate effervescent systems. In some embodiments, the disintegrant is partially pregeletanized maize starch.

In some embodiments, each granule comprises about 2 wt % to about 12 wt % of the disintegrant. In some embodiments, each granule comprises about 5 wt % to about 10 wt % of the disintegrant. In some embodiments, each granule comprises about 6 wt % to about 9 wt % of the disintegrant. In some embodiments, each granule comprises about 7 wt % to about 8 wt % of the disintegrant. In some embodiments, each granule comprises about 7.5 wt % of the disintegrant. In some embodiments, each granule comprises about 0.5 wt % to about 15 wt %, about 0.5 wt % to about 12.5 wt %, about 2.5 wt % to about 15 wt %, or about 2.5 wt % to about 12.5 wt % of the disintegrant (e.g., partially pregeletanized maize starch). In some embodiments, each granule comprises about 1.5 wt %, about 2.5 wt %, about 5 wt %, about 7 wt %, about 7.5 wt %, about 8 wt %, about 10 wt %, or about 12.5 wt % of the disintegrant (e.g., partially pregeletanized maize starch).

In some embodiments, the pharmaceutical dosage form comprises about 0.5 wt % to about 15 wt %, about 0.5 wt % to about 12.5 wt %, about 2.5 wt % to about 15 wt %, or about 2.5 wt % to about 12.5 wt % of the disintegrant (e.g., partially pregeletanized maize starch). In some embodiments, the pharmaceutical dosage form comprises about 1.5 wt %, about 2.5 wt %, about 5 wt %, about 7 wt %, about 7.5 wt %, about 8 wt %, about 10 wt %, or about 12.5 wt % of the disintegrant (e.g., partially pregeletanized maize starch).

In some embodiments, the at least one pharmaceutically acceptable carrier comprises a binder. As used herein, the term “binder” refers to a compound that can cause a composition to hold together. Exemplary binders include, but are not limited to, hydroxypropyl methylcellulose (also referred to as hypromellose), polyvinylpyrrolidone, natural gums (e.g., acacia gum), microcrystalline cellulose, methylcellulose, ethylcellulose, sucrose, starch, and gelatin. In some embodiments, the binder is hydroxypropyl methylcellulose.

In some embodiments, each granule comprises about 0.5 wt % to about 10 wt % of the binder. In some embodiments, each granule comprises about 2 wt % to about 8 wt % of the binder. In some embodiments, each granule comprises about 3 wt % to about 7 wt % of the binder. In some embodiments, each granule comprises about 4 wt % to about 6 wt % of the binder. In some embodiments, each granule comprises about 4.5 wt % to about 5.5 wt % of the binder. In some embodiments, each granule comprises about 5 wt % of the binder. In some embodiments, each granule comprises about 0.5 wt % to about 10 wt %, about 0.5 wt % to about 7.5 wt %, about 2.5 wt % to about 10 wt %, or about 2.5 wt % to about 7.5 wt % of the binder (e.g., hydroxypropyl methylcellulose). In some embodiments, each granule comprises about 1.5 wt %, about 2.5 wt %, about 4 wt %, about 5 wt %, about 6 wt %, or about 7.5 wt % of the binder (e.g., hydroxypropyl methylcellulose).

In some embodiments, the pharmaceutical dosage form comprises about 0.5 wt % to about 10 wt %, about 0.5 wt % to about 7.5 wt %, about 2.5 wt % to about 10 wt %, or about 2.5 wt % to about 7.5 wt % of the binder (e.g., hydroxypropyl methylcellulose). In some embodiments, the pharmaceutical dosage form comprises about 1.5 wt %, about 2.5 wt %, about 4 wt %, about 5 wt %, about 6 wt %, or about 7.5 wt % of the binder (e.g., hydroxypropyl methylcellulose).

In some embodiments, the at least one pharmaceutically acceptable carrier comprises a lubricant. As used herein, the term “lubricant” refers to a compound, e.g., an organic compound, that can reduce friction among the substances in a composition. Exemplary lubricants include, but are not limited to, magnesium stearate, calcium stearate, stearic acid (stearin), talc, starch, fumed silica, hydrogenated oil, polyethylene glycol, sodium stearyl fumarate, and glyceryl behenate. In some embodiments, the lubricant is magnesium stearate.

In some embodiments, each granule comprises about 0.5 wt % to about 5 wt % of the lubricant. In some embodiments, each granule comprises about 1 wt % to about 3 wt % of the lubricant. In some embodiments, each granule comprises about 2 wt % to about 2.8 wt % of the lubricant. In some embodiments, each granule comprises about 2.2 wt % to about 2.6 wt % of the lubricant. In some embodiments, each granule comprises about 2.3 wt % to about 2.5 wt % of the lubricant. In some embodiments, each granule comprises about 2.4 wt % to about 2.4 wt % of the lubricant. In some embodiments, each granule comprises about 2.5 wt % of the lubricant.

In some embodiments, each granule comprises about 0.5 wt % to about 5 wt %, about 0.5 wt % to about 4 wt %, about 1 wt % to about 5 wt %, or about 1 wt % to about 4 wt % of the lubricant (e.g., magnesium stearate). In some embodiments, each granule comprises about 1 wt %, about 2 wt %, about 3 wt %, or about 4 wt % of the lubricant (e.g., magnesium stearate). In some embodiments, pharmaceutical dosage form comprises about 0.5 wt % to about 5 wt %, about 0.5 wt % to about 4 wt %, about 1 wt % to about 5 wt %, or about 1 wt % to about 4 wt % of the lubricant (e.g., magnesium stearate). In some embodiments, the pharmaceutical dosage form comprises about 1 wt %, about 2 wt %, about 3 wt %, or about 4 wt % of the lubricant (e.g., magnesium stearate).

In some embodiments, each granule comprises about 5 wt % to about 40 wt % of valbenazine, or a pharmaceutically acceptable salt thereof (e.g., valbenazine ditosylate), based on the weight of the free base. In some embodiments, each granule comprises about 10 wt % to about 40 wt % of valbenazine, or a pharmaceutically acceptable salt thereof, based on the weight of the free base. In some embodiments, each granule comprises about 15 wt % to about 30 wt % of valbenazine, or a pharmaceutically acceptable salt thereof, based on the weight of the free base. In some embodiments, each granule comprises about 19 wt % to about 25 wt % of valbenazine, or a pharmaceutically acceptable salt thereof, based on the weight of the free base. In some embodiments, each granule comprises about 20 wt % to about 24 wt % of valbenazine, or a pharmaceutically acceptable salt thereof, based on the weight of the free base. In some embodiments, each granule comprises about 21 wt % to about 23 wt % of valbenazine, or a pharmaceutically acceptable salt thereof, based on the weight of the free base. In some embodiments, each granule comprises about 21.5 wt % to about 22.5 wt % of valbenazine, or a pharmaceutically acceptable salt thereof, based on the weight of the free base. In some embodiments, each granule comprises about 21.9 wt % to about 22.1 wt % of valbenazine, or a pharmaceutically acceptable salt thereof, based on the weight of the free base. In some embodiments, each granule comprises about 22 wt % of valbenazine, or a pharmaceutically acceptable salt thereof, based on the weight of the free base.

In some embodiments, each granule comprises about 5 wt % to about 40 wt % of valbenazine, or a pharmaceutically acceptable salt thereof (e.g., valbenazine ditosylate), based on the weight of the free base. In some embodiments, each granule comprises about 5 wt % to about 35 wt %, about 5 wt % to about 30 wt %, about 10 wt % to about 40 wt %, about 10 wt % to about 35 wt %, about 10 wt % to about 30 wt %, about 15 wt % to about 40 wt %, about 15 wt % to about 35 wt %, or about 15 wt % to about 30 wt % of valbenazine, or a pharmaceutically acceptable salt thereof (e.g., valbenazine ditosylate), based on the weight of the free base. In some embodiments, each granule comprises about 10 wt %, about 15 wt %, about 20 wt %, about 25 wt %, about 30 wt %, or about 35 wt % of valbenazine, or a pharmaceutically acceptable salt thereof (e.g., valbenazine ditosylate), based on the weight of the free base.

In some embodiments, each granule comprises about 20 wt % to about 50 wt % of valbenazine ditosylate. In some embodiments, each granule comprises about 10 wt % to about 60 wt %, about 25 wt % to about 60 wt %, about 25 wt % to about 50 wt %, about 10 wt % to about 40 wt %, about 25 wt % to about 35 wt %, or about 35 wt % to about 55 wt % of valbenazine ditosylate. In some embodiments, each granule comprises about 30 wt %, about 32 wt %, about 35 wt %, or about 40 wt % of valbenazine ditosylate.

In some embodiments, the pharmaceutical dosage form comprises about 5 wt % to about 40 wt % of valbenazine, or a pharmaceutically acceptable salt thereof (e.g., valbenazine ditosylate), based on the weight of the free base. In some embodiments, the pharmaceutical dosage form comprises about 5 wt % to about 35 wt %, about 5 wt % to about 30 wt %, about 10 wt % to about 40 wt %, about 10 wt % to about 35 wt %, about 10 wt % to about 30 wt %, about 15 wt % to about 40 wt %, about 15 wt % to about 35 wt %, or about 15 wt % to about 30 wt % of valbenazine, or a pharmaceutically acceptable salt thereof (e.g., valbenazine ditosylate), based on the weight of the free base. In some embodiments, the pharmaceutical dosage form comprises about 10 wt %, about 15 wt %, about 20 wt %, about 25 wt %, about 30 wt %, or about 35 wt % of valbenazine, or a pharmaceutically acceptable salt thereof (e.g., valbenazine ditosylate), based on the weight of the free base.

In some embodiments, the pharmaceutical dosage form comprises about 20 wt % to about 50 wt % of valbenazine ditosylate. In some embodiments, the pharmaceutical dosage form comprises about 10 wt % to about 60 wt %, about 25 wt % to about 60 wt %, about 25 wt % to about 50 wt %, about 10 wt % to about 40 wt %, about 25 wt % to about 35 wt %, or about 35 wt % to about 55 wt % of valbenazine ditosylate. In some embodiments, the pharmaceutical dosage form comprises about 30 wt %, about 32 wt %, about 35 wt %, or about 40 wt % of valbenazine ditosylate.

In some embodiments, valbenazine, or a pharmaceutically acceptable salt thereof, is valbenazine ditosylate.

In some embodiments, each granule comprises: (a) about 5 wt % to about 40 wt % valbenazine, or a pharmaceutically acceptable salt thereof, based on the weight of the free base; (b) about 25 wt % to about 65 wt % of a diluent; (c) about 0.5 wt % to about 15 wt % of a disintegrant; (d) about 0.5 wt % to about 10 wt % of a binder; and (e) about 0.5 wt % to about 5 wt % of a lubricant.

In some embodiments, each granule comprises: (a) about 30 wt % to about 50 wt % valbenazine ditosylate; (b) about 25 wt % to about 65 wt % of a diluent; (c) about 0.5 wt % to about 15 wt % of a disintegrant; (d) about 0.5 wt % to about 10 wt % of a binder; and (e) about 0.5 wt % to about 5 wt % of a lubricant.

In some embodiments, each granule comprises: (a) about 30 wt % to about 50 wt % valbenazine ditosylate; (b) about 15 wt % to about 35 wt % silicified microcrystalline cellulose; (c) about 10 wt % to about 30 wt % isomalt; (d) about 0.5 wt % to about 15 wt % partially pregeletanized maize starch; (e) about 0.5 wt % to about 10 wt % hydroxypropyl methylcellulose; and (f) about 0.5 wt % to about 5 wt % of magnesium stearate.

In some embodiments, each granule comprises: (a) about 5 wt % to about 40 wt % valbenazine, or a pharmaceutically acceptable salt thereof, based on the weight of the free base; (b) about 25 wt % to about 65 wt % of a diluent; (c) about 0.5 wt % to about 15 wt % of a disintegrant; (d) about 0.5 wt % to about 10 wt % of a binder; (e) about 0.5 wt % to about 5 wt % of a lubricant; and (f) about 3.5 wt % to about 15 wt % of the film-coating.

In some embodiments, each granule comprises: (a) about 30 wt % to about 50 wt % valbenazine ditosylate; (b) about 25 wt % to about 65 wt % of a diluent; (c) about 0.5 wt % to about 15 wt % of a disintegrant; (d) about 0.5 wt % to about 10 wt % of a binder; (d) about 0.5 wt % to about 5 wt % of a lubricant; and (e) about 3.5 wt % to about 15 wt % of a film-coating comprising: a film-forming polymer; a plasticizer; and a filler.

In some embodiments, each granule comprises: (a) about 30 wt % to about 50 wt % valbenazine ditosylate; (b) about 15 wt % to about 35 wt % silicified microcrystalline cellulose; (c) about 10 wt % to about 30 wt % isomalt; (d) about 0.5 wt % to about 15 wt % partially pregeletanized maize starch; (e) about 0.5 wt % to about 10 wt % hydroxypropyl methylcellulose; and (f) about 0.5 wt % to about 5 wt % magnesium stearate; and (g) about 3.5 wt % to about 15 wt % of a film-coating comprising: poly(vinyl alcohol); polyethylene glycol; and talc.

In some embodiments, each granule comprises: (a) about 33 wt % to about 40 wt % valbenazine ditosylate; (b) about 20 wt % to about 25 wt % silicified microcrystalline cellulose; (c) about 16 wt % to about 20 wt % isomalt; (d) about 6 wt % to about 8 wt % partially pregeletanized maize starch; (e) about 4 wt % to about 5 wt % hydroxypropyl methylcellulose; (f) about 2 wt % to about 2.5 wt % magnesium stearate; and (g) about 8 wt % to about 11 wt % of the film-coating.

In some embodiments, each granule comprises: (a) about 33 wt % to about 40 wt % valbenazine ditosylate; (b) about 20 wt % to about 25 wt % silicified microcrystalline cellulose; (c) about 16 wt % to about 20 wt % isomalt; (d) about 6 wt % to about 8 wt % partially pregeletanized maize starch; (e) about 4 wt % to about 5 wt % hydroxypropyl methylcellulose; (f) about 2 wt % to about 2.5 wt % magnesium stearate; and (e) about 8 wt % to about 11 wt % of the film-coating, wherein the film-coating comprises polyvinyl alcohol, polyethylene glycol, talc, and titanium dioxide.

In some embodiments, each granule comprises: (a) about 33 wt % to about 40 wt % valbenazine ditosylate; (b) about 20 wt % to about 25 wt % silicified microcrystalline cellulose; (c) about 16 wt % to about 20 wt % isomalt; (d) about 6 wt % to about 8 wt % partially pregeletanized maize starch; (e) about 4 wt % to about 5 wt % hydroxypropyl methylcellulose; (f) about 2 wt % to about 2.5 wt % magnesium stearate; and (g) about 8 wt % to about 11 wt % OPADRY® II.

In some embodiments, each granule comprises: (a) about 34.5 wt % to about 38.2 wt % valbenazine ditosylate; (b) about 22.6 wt % to about 24 wt % silicified microcrystalline cellulose; (c) about 17.3 wt % to about 19.2 wt % isomalt; (d) about 6.5 wt % to about 7.2 wt % partially pregeletanized maize starch; (e) about 4.4 wt % to about 5 wt % hydroxypropyl methylcellulose; (f) about 2 wt % to about 2.3 wt % magnesium stearate; and (g) about 8.2 wt % to about 10.5 wt % of the film-coating.

In some embodiments, each granule comprises: (a) about 34.5 wt % to about 38.2 wt % valbenazine ditosylate; (b) about 22.6 wt % to about 24 wt % silicified microcrystalline cellulose; (c) about 17.3 wt % to about 19.2 wt % isomalt; (d) about 6.5 wt % to about 7.2 wt % partially pregeletanized maize starch; (e) about 4.4 wt % to about 5 wt % hydroxypropyl methylcellulose; (f) about 2 wt % to about 2.3 wt % magnesium stearate; and (g) about 8.2 wt % to about 10.5 wt % of the film-coating, wherein the film-coating comprises polyvinyl alcohol, polyethylene glycol, talc, and titanium dioxide.

In some embodiments, each granule comprises: (a) about 34.5 wt % to about 38.2 wt % valbenazine ditosylate; (b) about 22.6 wt % to about 24 wt % silicified microcrystalline cellulose; (c) about 17.3 wt % to about 19.2 wt % isomalt; (d) about 6.5 wt % to about 7.2 wt % partially pregeletanized maize starch; (e) about 4.4 wt % to about 5 wt % hydroxypropyl methylcellulose; (f) about 2 wt % to about 2.3 wt % magnesium stearate; and (g) about 8.2 wt % to about 10.5 wt % OPADRY® II.

In some embodiments, the pharmaceutical dosage form comprises: (a) about 5 wt % to about 40 wt % valbenazine, or a pharmaceutically acceptable salt thereof, based on the weight of the free base; (b) about 25 wt % to about 65 wt % of a diluent; (c) about 0.5 wt % to about 15 wt % of a disintegrant; (d) about 0.5 wt % to about 10 wt % of a binder; and (e) about 0.5 wt % to about 5 wt % of a lubricant.

In some embodiments, the pharmaceutical dosage form comprises: (a) about 30 wt % to about 50 wt % valbenazine ditosylate; (b) about 25 wt % to about 65 wt % of a diluent; (c) about 0.5 wt % to about 15 wt % of a disintegrant; (d) about 0.5 wt % to about 10 wt % of a binder; and (e) about 0.5 wt % to about 5 wt % of a of a lubricant.

In some embodiments, the pharmaceutical dosage form comprises: (a) about 30 wt % to about 50 wt % valbenazine ditosylate; (b) about 15 wt % to about 35 wt % silicified microcrystalline cellulose; (c) about 10 wt % to about 30 wt % isomalt; (d) about 0.5 wt % to about 15 wt % partially pregeletanized maize starch; (e) about 0.5 wt % to about 10 wt % hydroxypropyl methylcellulose; and (f) about 0.5 wt % to about 5 wt % of magnesium stearate.

In some embodiments, the pharmaceutical dosage form comprises: (a) about 5 wt % to about 40 wt % valbenazine, or a pharmaceutically acceptable salt thereof, based on the weight of the free base; (b) about 25 wt % to about 65 wt % of a diluent; (c) about 0.5 wt % to about 15 wt % of a disintegrant; (d) about 0.5 wt % to about 10 wt % of a binder; (e) about 0.5 wt % to about 5 wt % of a lubricant; and (f) about 3.5 wt % to about 15 wt % of the film-coating.

In some embodiments, the pharmaceutical dosage form comprises: (a) about 30 wt % to about 50 wt % valbenazine ditosylate; (b) about 25 wt % to about 65 wt % of a diluent; (c) about 0.5 wt % to about 15 wt % of a disintegrant; (d) about 0.5 wt % to about 10 wt % of a binder; (d) about 0.5 wt % to about 5 wt % of a lubricant; and (e) about 3.5 wt % to about 15 wt % of a film-coating comprising: a film-forming polymer; a plasticizer, and a filler.

In some embodiments, the pharmaceutical dosage form comprises: (a) about 30 wt % to about 50 wt % valbenazine ditosylate; (b) about 15 wt % to about 35 wt % silicified microcrystalline cellulose; (c) about 10 wt % to about 30 wt % isomalt; (d) about 0.5 wt % to about 15 wt % partially pregeletanized maize starch; (e) about 0.5 wt % to about 10 wt % hydroxypropyl methylcellulose; and (f) about 0.5 wt % to about 5 wt % magnesium stearate; and (g) about 3.5 wt % to about 15 wt % of a film-coating comprising: poly(vinyl alcohol); polyethylene glycol; and talc.

In some embodiments, the pharmaceutical dosage form comprises: (a) about 33 wt % to about 40 wt % valbenazine ditosylate; (b) about 20 wt % to about 25 wt % silicified microcrystalline cellulose; (c) about 16 wt % to about 20 wt % isomalt; (d) about 6 wt % to about 8 wt % partially pregeletanized maize starch; (e) about 4 wt % to about 5 wt % hydroxypropyl methylcellulose; (f) about 2 wt % to about 2.5 wt % magnesium stearate; and (g) about 8 wt % to about 11 wt % of the film-coating.

In some embodiments, the pharmaceutical dosage form comprises: (a) about 33 wt % to about 40 wt % valbenazine ditosylate; (b) about 20 wt % to about 25 wt % silicified microcrystalline cellulose; (c) about 16 wt % to about 20 wt % isomalt; (d) about 6 wt % to about 8 wt % partially pregeletanized maize starch; (e) about 4 wt % to about 5 wt % hydroxypropyl methylcellulose; (f) about 2 wt % to about 2.5 wt % magnesium stearate; and (e) about 8 wt % to about 11 wt % of the film-coating, wherein the film-coating comprises polyvinyl alcohol, polyethylene glycol, talc, and titanium dioxide.

In some embodiments, the pharmaceutical dosage form comprises: (a) about 33 wt % to about 40 wt % valbenazine ditosylate; (b) about 20 wt % to about 25 wt % silicified microcrystalline cellulose; (c) about 16 wt % to about 20 wt % isomalt; (d) about 6 wt % to about 8 wt % partially pregeletanized maize starch; (e) about 4 wt % to about 5 wt % hydroxypropyl methylcellulose; (f) about 2 wt % to about 2.5 wt % magnesium stearate; and (g) about 8 wt % to about 11 wt % OPADRY® II.

In some embodiments, the pharmaceutical dosage form comprises: (a) about 34.5 wt % to about 38.2 wt % valbenazine ditosylate; (b) about 22.6 wt % to about 24 wt % silicified microcrystalline cellulose; (c) about 17.3 wt % to about 19.2 wt % isomalt; (d) about 6.5 wt % to about 7.2 wt % partially pregeletanized maize starch; (e) about 4.4 wt % to about 5 wt % hydroxypropyl methylcellulose; (f) about 2 wt % to about 2.3 wt % magnesium stearate; and (g) about 8.2 wt % to about 10.5 wt % of the film-coating.

In some embodiments, the pharmaceutical dosage form comprises: (a) about 34.5 wt % to about 38.2 wt % valbenazine ditosylate; (b) about 22.6 wt % to about 24 wt % silicified microcrystalline cellulose; (c) about 17.3 wt % to about 19.2 wt % isomalt; (d) about 6.5 wt % to about 7.2 wt % partially pregeletanized maize starch; (e) about 4.4 wt % to about 5 wt % hydroxypropyl methylcellulose; (f) about 2 wt % to about 2.3 wt % magnesium stearate; and (g) about 8.2 wt % to about 10.5 wt % of the film-coating, wherein the film-coating comprises polyvinyl alcohol, polyethylene glycol, talc, and titanium dioxide.

In some embodiments, the pharmaceutical dosage form comprises: (a) about 34.5 wt % to about 38.2 wt % valbenazine ditosylate; (b) about 22.6 wt % to about 24 wt % silicified microcrystalline cellulose; (c) about 17.3 wt % to about 19.2 wt % isomalt; (d) about 6.5 wt % to about 7.2 wt % partially pregeletanized maize starch; (e) about 4.4 wt % to about 5 wt % hydroxypropyl methylcellulose; (f) about 2 wt % to about 2.3 wt % magnesium stearate; and (g) about 8.2 wt % to about 10.5 wt % OPADRY® II.

In some embodiments, each granule comprises an average amount of valbenazine, or a pharmaceutically acceptable salt thereof, of about 1.5 mg to about 2.5 mg, based on the weight of the free base. In some embodiments, each granule comprises an average amount of valbenazine, or a pharmaceutically acceptable salt thereof, of about 1.8 mg to about 2.2 mg, based on the weight of the free base. In some embodiments, each granule comprises an average amount of valbenazine, or a pharmaceutically acceptable salt thereof, of about 1.85 mg to about 2.15 mg, based on the weight of the free base. In some embodiments, each granule comprises an average amount of valbenazine, or a pharmaceutically acceptable salt thereof, of about 1.9 mg to about 2.1 mg, based on the weight of the free base. In some embodiments, each granule comprises an average amount of valbenazine, or a pharmaceutically acceptable salt thereof, of about 1.95 mg to about 2.05 mg, based on the weight of the free base. In some embodiments, each granule comprises an average amount of valbenazine, or a pharmaceutically acceptable salt thereof, of about 2 mg, based on the weight of the free base.

In some embodiments, each granule comprises an average amount of valbenazine ditosylate of about 3 mg to about 4.5 mg. In some embodiments, each granule comprises an average amount of valbenazine ditosylate of about 3.4 mg to about 4 mg. In some embodiments, each granule comprises an average amount of valbenazine ditosylate of about 3.5 mg to about 3.9 mg. In some embodiments, each granule comprises an average amount of valbenazine ditosylate of about 3.6 mg to about 3.8 mg. In some embodiments, each granule comprises an average amount of valbenazine ditosylate of about 3.7 mg.

In some embodiments, the pharmaceutical dosage form is a capsule. In some embodiments, the capsule is a size 00 or smaller. In some embodiments, the capsule is a size 00. In some embodiments, the capsule is a size 0 or smaller. In some embodiments, the capsule is a size 0. In some embodiments, the capsule is a size 1 or smaller. In some embodiments, the capsule is a size 1. In some embodiments, the capsule is a size 2. In some embodiments, the capsule is a size 2 or smaller. In some embodiments, the capsule is a sprinkle capsule. As used herein, “capsule size” refers to the internationally accepted numbering system for capsule sizes used in approved U.S. drug products.

Also provided herein is a unit dosage form comprising the pharmaceutical dosage form as described herein, wherein the valbenazine, or a pharmaceutically acceptable salt thereof, is present in an amount of about 10 mg to about 200 mg, based on the weight of the free base. The term “unit dosage form” refers to a physically discrete unit suitable as a unitary dosage for human subjects and other mammals, each unit containing a predetermined quantity of active material calculated to produce the desired therapeutic effect, in association with a suitable pharmaceutical carrier. In some embodiments, the valbenazine, or a pharmaceutically acceptable salt thereof, in the unit dosage form is present in an amount of about 20 mg to about 100 mg, based on the weight of the free base. In some embodiments, the valbenazine, or a pharmaceutically acceptable salt thereof, in the unit dosage form is present in an amount of about 20 mg to about 80 mg, based on the weight of the free base. In some embodiments, valbenazine, or a pharmaceutically acceptable salt thereof, in the unit dosage form is present in an amount of about 10 mg, based on the weight of the free base. In some embodiments, valbenazine, or a pharmaceutically acceptable salt thereof, in the unit dosage form is present in an amount of about 20 mg, based on the weight of the free base. In some embodiments, valbenazine, or a pharmaceutically acceptable salt thereof, in the unit dosage form is present in an amount of about 40 mg, based on the weight of the free base. In some embodiments, valbenazine, or a pharmaceutically acceptable salt thereof, in the unit dosage form is present in an amount of about 60 mg, based on the weight of the free base. In some embodiments, valbenazine, or a pharmaceutically acceptable salt thereof, in the unit dosage form is present in an amount of about 80 mg, based on the weight of the free base. In some embodiments, valbenazine, or a pharmaceutically acceptable salt thereof, in the unit dosage form is present in an amount of about 100 mg, based on the weight of the free base.

In some embodiments, valbenazine, or a pharmaceutically acceptable salt thereof, in the unit dosage form is present in an amount of about 20 mg to about 200 mg, about 20 mg to about 160 mg, about 40 mg to about 200 mg, about 40 mg to about 160 mg, about 60 mg to about 200 mg, or about 60 mg to about 160 mg, based on the weight of the free base. In some embodiments, valbenazine, or a pharmaceutically acceptable salt thereof, in the unit dosage form is present in an amount of about 20 mg, about 40 mg, about 60 mg, about 80 mg, about 100 mg, about 120 mg, about 140 mg, or about 160 mg, based on the weight of the free base.

In some embodiments, the unit dosage form is a capsule. In some embodiments, the capsule is a size 00 or smaller. In some embodiments, the capsule is a size 00. In some embodiments, the capsule is a size 0 or smaller. In some embodiments, the capsule is a size 0. In some embodiments, the capsule is a size 1 or smaller. In some embodiments, the capsule is a size 1. In some embodiments, the capsule is a size 2 or smaller. In some embodiments, the capsule is a size 2. In some embodiments, the capsule is a sprinkle capsule. As used herein, “capsule size” refers to the internationally accepted numbering system for capsule sizes used in approved U.S. drug products.

### Processes for Preparing the Pharmaceutical Dosage Forms

Provided is a process for preparing granules comprising an amount of valbenazine, or a pharmaceutically acceptable salt thereof, the process comprising:
(1) roller compacting a blend comprising milled isomalt (item 3), milled valbenazine ditosylate (item 1), milled silicified microcrystalline cellulose (item 2), milled partially pregelatinized maize starch (item 4), milled hypromellose (item 5), and sieved magnesium stearate (item 6) to obtain roller compacted ribbon material;
(2) milling the roller compacted ribbon material;
(3) blending additional sieved magnesium stearate and the milled roller compacted ribbon material of step (2) to obtain a final blend; and
(4) pressing the final blend to obtain granules.

In some embodiments, provided is a process for preparing granules comprising an amount of valbenazine, or a pharmaceutically acceptable salt thereof, the process comprising:
(1) blending isomalt (item 3), valbenazine ditosylate (item 1), and silicified microcrystalline cellulose (item 2);
(2) milling the mixture of step (1) and transferring the milled material to a bin;
(3) blending the mixture of step (2);
(4) milling partially pregelatinized maize starch (item 4) and hypromellose (item 5) and adding the milled mixture comprising item 4 and item 5 to the mixture of step (3);
(5) blending the mixture of step (4);
(6) adding sieved magnesium stearate (item 6) to the mixture of step (5);
(7) blending the mixture of step (6);
(8) roller compacting the mixture of step (7) to obtain roller compacted ribbon material;
(9) milling the roller compacted ribbon material;
(10) blending additional sieved magnesium stearate and the milled roller compacted ribbon material of step (9) to obtain a final blend; and
(11) pressing the final blend to obtain granules.

Roller compacting may be carried out in any suitable apparatus such as, for example, a Fitzmill IR 220 Roller Compactor, to provide ribbon material. The roller-compacted ribbon is milled by passing through a Fitzmill to provide granulated material.

The pressing of the final blend is carried out in a suitable tablet press such as, for example, a Fette 52i Press. In some embodiments, after the final blend is pressed to obtain granules, the process further comprises coating the granules with a film-coating.

The present disclosure further provides a process for preparing film-coated granules of the pharmaceutical dosage form disclosed herein, comprising coating a granule core with a film-coating.

In some embodiments, the film-coating comprises a film-forming polymer and one or more of a plasticizer, a filler, and a pigment/opacifier.

In some embodiments, the film-coating comprises a film-forming polymer. In some embodiments, the film-coating comprises a film-forming polymer, and the film-forming polymer is about 25 wt % to about 55 wt %, about 25 wt % to about 45 wt %, about 35 wt % to about 55 wt % or about 35 wt % to about 45 wt % of the weight of the film-coating. In some embodiments, the film-coating comprises a film-forming polymer, and the film-forming polymer is about 30 wt %, about 35 wt %, about 40 wt %, about 45 wt %, or about 50 wt % of the weight of the film-coating. In some embodiments, the film-forming polymer is poly(vinyl alcohol).

In some embodiments, the film-coating comprises a plasticizer. In some embodiments, the film-coating comprises a plasticizer, and the plasticizer is about 5 wt % to about 30 wt % about 5 wt % to about 25 wt, about 10 wt % to about 30 wt %, or about 10 wt % to about 25 wt % of the weight of the film-coating. In some embodiments, the film-coating comprises a plasticizer, and the plasticizer is about 10 wt %, about 15 wt %, about 20 wt %, or about 25 wt % of the weight of the film-coating. In some embodiments, the plasticizer is polyethylene glycol.

In some embodiments, the film-coating comprises a filler, and the filler is about 5 wt % to about 45 wt % about 5 wt % to about 20 wt %, about 10 wt % to about 45 wt %, or about 10 wt % to about 20 wt % of the weight of the film-coating. In some embodiments, the film-coating comprises a filler, and the filler is about 10 wt %, about 15 wt %, about 20 wt %, about 25 wt %, about 30 wt %, about 35 wt %, or about 40 wt % of the weight of the film-coating. In some embodiments, the filler is talc.

In some embodiments, the film-coating comprises a pigment/opacifier, and the pigment/opacifier is about 5 wt % to about 40 wt %, about 10 wt % to about 40 wt %, 20 wt % to about 40 wt %, or about 20 wt % to about 30 wt % of the weight of the film-coating. In some embodiments, the film-coating comprises a pigment/opacifier, and the pigment/opacifier is about 5 wt %, about 10 wt %, about 15 wt %, about 20 wt %, about 25 wt %, about 30 wt %, or about 35 wt % of the weight of the film-coating. In some embodiments, the pigment/opacifier is titanium dioxide.

In some embodiments, the film-coating comprises: (a) about 25 wt % to about 55 wt % of a film-forming polymer; (b) about 5 wt % to about 30 wt % of a plasticizer; (c) about 5 wt % to about 45 wt % of a filler; and (d) up to about 40 wt % of a pigment/opacifier; based on the weight of the film-coating. In some embodiments, the film-coating comprises: (a) about 25 wt % to about 55 wt % of poly(vinyl alcohol); (b) about 5 wt % to about 30 wt % of polyethylene glycol; (c) about 5 wt % to about 45 wt % of talc; and (d) up to about 40 wt % of titanium dioxide; based on the weight of the film-coating.

In some embodiments, the film-coating comprises OPADRY® II. In some embodiments, the film-coating comprises OPADRY® II. In some embodiments, the film-coating consists essentially of OPADRY® II. In some embodiments, the film-coating is OPADRY® II.

In some embodiments, the film-coating makes up about 3.5 wt % to about 15 wt % of the weight of each granule. In some embodiments, the film-coating makes up about 3.5 wt % to about 12.5 wt %, about 5 wt % to about 15 wt %, or about 5 wt % to about 12.5 wt % of the weight of each granule. In some embodiments, the film-coating makes up about 5 wt %, about 7.5 wt %, about 10 wt %, or about 12.5 wt % of the weight of each granule.

In some embodiments, coating the granule core with a film-coating comprises spraying a mixture comprising water and the film-coating onto the surface of the granule core, and contacting the granules with a process gas having an elevated temperature, such as a temperature of at least about 40° C. As used herein, the term “process gas” refers to a gas or mixture of gases suitable for use in a process described herein.

In some embodiments, coating the granules with a film-coating comprises fluidizing the granules using a process gas having a temperature of at least about 40° C., at least about 45° C., or at least about 50° C., and spraying a mixture comprising water and the film-coating onto the surface of the fluidized granules. In some embodiments, the temperature is about 40° C. to about 65° C. In some embodiments, the temperature is about 45° C. to about 60° C. In some embodiments, the temperature is about 50° C. to about 54° C. In some embodiments, the process gas comprises one or more inert gases (e.g., nitrogen). In some embodiments, the process gas comprises air.

The granule core can be formed by compressing a precursor composition comprising valbenazine or a pharmaceutically acceptable salt thereof, wherein the precursor composition comprises particles have an average diameter less than about 1 mm.

In some embodiments, compressing the precursor composition comprises pressing the precursor composition in a tablet press. Compressing the precursor composition can form the granule core described herein.

In some embodiments, compressing the precursor composition forms granules having an average diameter of about 1.5 mm to about 5 mm, about 2 mm to about 3 mm, about 2.15 mm to about 2.25 mm, about 2.18 mm to about 2.23 mm, or about 2.19 mm to about 2.21 mm.

In some embodiments, compressing the precursor composition forms granules having an average diameter of about 1 mm to about 5 mm, about 1 mm to about 4 mm, about 1 mm to about 3.5 mm, about 1.2 mm to about 5 mm, about 1.2 mm to about 4 mm, or about 1.2 mm to about 3.5 mm. In some embodiments, compressing the precursor composition forms granules having an average diameter of about 1.2 mm, about 1.4 mm, about 1.6 mm, about 1.8 mm, about 2 mm, about 2.2 mm, about 2.4 mm, about 2.6 mm, about 2.8 mm, or about 3 mm.

In some embodiments, compressing the precursor composition forms granules having an average diameter of about 1.2 mm to about 4 mm, an average diameter variation of no more than 10% from the average diameter, and an average density of about 0.5 g/cm³ to about 2.5 g/cm³. In some embodiments, compressing the precursor composition forms granules having an average diameter of about 1.2 mm to about 4 mm, an average diameter variation of no more than 10% from the average diameter, and an average density of about 0.75 g/cm³ to about 2.5 g/cm³. In some embodiments, compressing the precursor composition forms granules having an average diameter of about 1.2 mm to about 3.5 mm, an average diameter variation of no more than 10% from the average diameter, and an average density of about 0.75 g/cm³ to about 2.5 g/cm³. In some embodiments, compressing the precursor composition forms granules having an average diameter of about 1.2 mm to about 3.5 mm, an average diameter variation of no more than 10% from the average diameter, and an average density of about 1 g/cm³ to about 2 g/cm³.

In some embodiments, compressing the precursor composition forms granules having an average diameter of at most about 2.5 mm, a d99 particle diameter distribution of at most about 2.8 mm, and an average density of about 0.75 g/cm³ to about 2.5 g/cm³. In some embodiments, compressing the precursor composition forms granules having an average diameter of at most about 2.5 mm, a d99 particle diameter distribution of at most about 2.8 mm, and an average density of about 1 g/cm³ to about 2.5 g/cm³. In some embodiments, compressing the precursor composition forms granules having an average diameter of at most about 2.2 mm, a d99 particle diameter distribution of at most about 2.5 mm, and an average density of about 0.75 g/cm³ to about 2 g/cm³. In some embodiments, compressing the precursor composition forms granules having an average diameter of at most about 2.2 mm, a d99 particle diameter distribution of at most about 2.5 mm, and an average density of about 1 g/cm³ to about 2 g/cm³.

In some embodiments, compressing the precursor composition forms granules having an average hardness of about 1 kp to about 2.5 kp. In some embodiments, compressing the precursor composition forms granules having an average hardness of about 1 kp to about 2 kp, about 1.2 kp to about 2.5 kp, or about 1.2 kp to about 2 kp. In some embodiments, compressing the precursor composition forms granules having an average hardness of about 1 kp, about 1.2 kp, about 1.4 kp, about 1.6 kp, about 1.8 kp, or about 2 kp.

The precursor composition can be formed by compacting a blend of valbenazine, or a pharmaceutically acceptable salt thereof, and at least one pharmaceutically acceptable carrier (e.g., a diluent, a disintegrant, a binder, or a lubricant); and milling the compacted blend.

In some embodiments, the precursor composition comprises about 5 wt % to about 40 wt % of valbenazine, or a pharmaceutically acceptable salt thereof (e.g., valbenazine ditosylate), based on the weight of the free base. In some embodiments, the precursor composition comprises about 5 wt % to about 35 wt %, about 5 wt % to about 30 wt %, about 10 wt % to about 40 wt %, about 10 wt % to about 35 wt %, about 10 wt % to about 30 wt %, about 15 wt % to about 40 wt %, about 15 wt % to about 35 wt %, or about 15 wt % to about 30 wt % of valbenazine, or a pharmaceutically acceptable salt thereof (e.g., valbenazine ditosylate), based on the weight of the free base. In some embodiments, the precursor composition comprises about 10 wt %, about 15 wt %, about 20 wt %, about 25 wt %, or about 30 wt % of valbenazine, or a pharmaceutically acceptable salt thereof (e.g., valbenazine ditosylate), based on the weight of the free base.

In some embodiments, the precursor composition comprises at least one pharmaceutically acceptable carrier, such as, one or more of a diluent, a disintegrant, a binder, and a lubricant (e.g., selected to provide the granule core described herein). In some embodiments, the precursor compositions described herein comprise about 5 wt % to about 40 wt % valbenazine, or a pharmaceutically acceptable salt thereof, based on the weight of the free base, about 25 wt % to about 65 wt % of a diluent, about 0.5 wt % to about 15 wt % of a disintegrant, about 0.5 wt % to about 10 wt % of a binder, and about 0.5 wt % to about 5 wt % of a lubricant.

In some embodiments, the precursor compositions described herein comprise about 30 wt % to about 50 wt % valbenazine ditosylate, about 25 wt % to about 65 wt % of a diluent, about 0.5 wt % to about 15 wt % of a disintegrant, about 0.5 wt % to about 10 wt % of a binder, and about 0.5 wt % to about 5 wt % of a lubricant.

In some embodiments, the precursor compositions described herein comprise about 30 wt % to about 50 wt % valbenazine ditosylate, about 15 wt % to about 35 wt % silicified microcrystalline cellulose, about 10 wt % to about 30 wt % isomalt, about 0.5 wt % to about 15 wt % partially pregeletanized maize starch; about 0.5 wt % to about 10 wt % hydroxypropyl methylcellulose, and about 0.5 wt % to about 5 wt % magnesium stearate.

In some embodiments, the precursor compositions described herein comprise (a) about 33 wt % to about 40 wt % valbenazine ditosylate; (b) about 20 wt % to about 25 wt % silicified microcrystalline cellulose; (c) about 16 wt % to about 20 wt % isomalt; (d) about 6 wt % to about 8 wt % partially pregeletanized maize starch; (e) about 4 wt % to about 5 wt % hydroxypropyl methylcellulose; and (f) about 2 wt % to about 2.5 wt % magnesium stearate.

In some embodiments, the precursor compositions described herein comprise about 40 wt % valbenazine ditosylate, about 25 wt % silicified microcrystalline cellulose, about 20 wt % isomalt, about 7.5 wt % partially pregeletanized maize starch, about 5 wt % hydroxypropyl methylcellulose, and about 2.5 wt % magnesium stearate.

In some embodiments, the precursor compositions described herein comprise (a) about 34.5 wt % to about 38.2 wt % valbenazine ditosylate; (b) about 22.6 wt % to about 24 wt % silicified microcrystalline cellulose; (c) about 17.3 wt % to about 19.2 wt % isomalt; (d) about 6.5 wt % to about 7.2 wt % partially pregeletanized maize starch; (e) about 4.4 wt % to about 5 wt % hydroxypropyl methylcellulose; and (f) about 2 wt % to about 2.3 wt % magnesium stearate.

In some embodiments, the precursor composition comprises particles having an average particle diameter of at most about 0.9 mm. In some embodiments, the precursor composition comprises particles having an average particle diameter of about 1 μm to about 0.9 mm, about 10 μm to about 0.9 mm, about 50 μm to about 0.9 mm, about 1 μm to about 0.8 mm, about 10 μm to about 0.8 mm, or about 50 μm to about 0.8 mm. In some embodiments, the precursor composition comprises particles having an average particle diameter of about 50 μm, about 100 μm, about 200 μm, about 300 μm, about 400 μm, or about 500 μm.

In some embodiments, the precursor composition has a d99 particle diameter distribution of at most about 0.9 mm. In some embodiments, the precursor composition has a d99 particle diameter distribution of about 50 μm to about 0.9 mm, or about 50 μm to about 0.8 mm. In some embodiments, the precursor composition has a d90 particle diameter distribution of at most about 500 μm, at most about 250 μm, or at most about 100 μm. In some embodiments, the precursor composition has a d90 particle diameter distribution of about 10 μm to about 500 μm, about 10 μm to about 250 μm, or about 10 μm to about 100 μm.

### Definitions

As used herein, and unless otherwise specified, the term “about,” when used in connection with a numeric value or range of values which is provided to describe a particular composition, salt or solid form, e.g., a specific size measurement, a specific temperature or temperature range, such as, for example, that describing a melting, dehydration, or glass transition; a mass change, such as, for example, a mass change as a function of temperature or humidity; a solvent or water content, in terms of, for example, mass or a percentage; or a peak position, such as, for example, in analysis by, for example, ¹³C NMR, DSC, TGA and XRPD; indicate that the value or range of values may deviate to an extent deemed reasonable to one of ordinary skill in the art while still describing the particular solid form. Specifically, the term “about”, when used in this context, indicates that the numeric value or range of values may vary by 5%, 4%, 3%, 2%, or 1% of the recited value or range of values while still describing the particular solid form. In some embodiments, the term “about” indicates that the numeric value or range of values may vary by 5%. As used herein, the terms “blend,” “blending,” and “blended” refer to combining or mixing different substance to obtain a mixture. The resulting blended mixture can be homogeneous.

As used herein, the diameter of a granule described herein refers to the largest dimension thereof (e.g., a diameter of a spherical particle, a length of an ovoid particle, the greater of the height and diameter of a cylindrical particle, etc.). As used herein, a d99 particle diameter distribution refers to the value of the particle diameter (e.g., the diameter of a granule) at which about 99% of the particles in a sample have a value that is lower than the d99 value, and about 1% of the particles in the sample have a value that is higher than the d99 value. The average diameter and particle diameter distribution of granules described herein can be measured using conventional particle size analysis techniques (e.g., sieve analysis, laser diffraction analysis, or microscope counting). Additional methods of measurement are described in U.S. Pharmacopeia (“USP”) 905 (Uniformity of Dosage Units (2016)) and USP 429 (Light Diffraction Measurement of Particle Size (2016)), each of which is incorporated herein by reference for all purposes.

As used herein, the term “diameter variation” indicates the maximum amount that the diameter of a particle in a distribution varies from the average diameter of the distribution. For example, for a sample of granules having a diameter variation of no more than 20%, the diameter of each granule in the sample is no less than 20% and no more than 20% of the indicated average diameter of the granules.

As used herein, the “average hardness” of a granule refers to a measure of the breaking point and structural integrity of a granule. In particular, the average hardness refers to the average amount of force that is required to be applied to a granule in order to cause the granule to begin to fracture. Average hardness can be measured using conventions practices in the art, such as compression testing, or using devices such as a Monsanto tester or a Pfizer tester.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

Various pharmaceutically acceptable carriers can be used in the formulations described herein. As used herein, “pharmaceutically acceptable carriers” refers to a pharmaceutically-acceptable material, composition, or vehicle, such as a liquid or solid filler, diluent, solvent, or encapsulating material. Pharmaceutically acceptable carriers are also known as “pharmaceutically acceptable excipients.” Carriers are generally safe, non-toxic and neither biologically nor otherwise undesirable and include carriers that are acceptable for veterinary use as well as human pharmaceutical use. In one embodiment, each component is “pharmaceutically acceptable” as defined herein. See, e.g., Remington: The Science and Practice of Pharmacy, 21st ed; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19, and in Stahl et al., Handbook of Pharmaceutical Salts: Properties, Selection, and Use, (Wiley, 2002).

As used herein, the term “individual,” “patient,” or “subject” used interchangeably, refers to any animal, including mammals, preferably mice, rats, monkeys, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor, or other clinician. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

### Methods of Use

The disclosure is also directed to a method of treating a neurological or psychiatric disease or disorder in a patient in need thereof, comprising administering to the patient a therapeutically effective amount of the pharmaceutical dosage form or the unit dosage form, both as described herein.

In some embodiments, the patient is suffering from dysphagia or another swallowing disorder. In some embodiments, the patient is suffering from dysphagia. In some embodiments, the patient has dysphagia.

In some embodiments, the patient is a pediatric patient, such as a child 12 years of age or under, 6 years of age or under, 5 years of age or under, 4 years of age or under, 3 years of age or under, 2 years of age or under, or 1 year of age or under. In some embodiments, the pediatric patient's age is about 6 months to about 5 years, about 6 months to about 4 years, about 6 months to about 3 years, about 6 months to about 2 years, or about 6 months to about 1 year.

In some embodiments, the patient is a geriatric patient, for example, a geriatric patient suffering from dysphagia or other swallowing disorder. In some embodiments, the geriatric patient is at least 50 years old, at least 60 years old, at least 70 years old, or at least 80 years old.

Disclosed is a method of administering valbenazine, or a pharmaceutically acceptable salt thereof, to a patient in need thereof, comprising:
(a) providing the pharmaceutical dosage form or the unit dosage form, both as described herein;
(b) sprinkling the plurality of granules on soft food; and
(c) administering the soft food orally.

In some embodiments, the soft food is chosen from applesauce, yogurt, pudding, ice cream, baby food, and a soy or grain-based product.

Disclosed is a method of administering valbenazine, or a pharmaceutically acceptable salt thereof, to a patient in need thereof, comprising: orally administering the pharmaceutical dosage form or the unit dosage, both as described herein.

Disclosed is a method of administering valbenazine, or a pharmaceutically acceptable salt thereof, to a patient in need thereof, comprising: orally administering a capsule containing the pharmaceutical dosage form or the unit dosage, both as described herein.

In some embodiments, each granule is suitable for oral administration.

In some embodiments, orally administering a pharmaceutical composition described herein includes sprinkling the composition onto food (e.g., a soft food).

In some embodiments, the neurological or psychiatric disease or disorder is a hyperkinetic movement disorder, mood disorder, bipolar disorder, schizophrenia, schizoaffective disorder, mania in mood disorder, depression in mood disorder, treatment-refractory obsessive compulsive disorder, neurological dysfunction associated with Lesch-Nyhan syndrome, agitation associated with Alzheimer's disease, Fragile X syndrome or Fragile X-associated tremor-ataxia syndrome, autism spectrum disorder, Rett syndrome, or chorea-acanthocytosis.

In some embodiments, the neurological or psychiatric disease or disorder is a hyperkinetic movement disorder. In some embodiments, the hyperkinetic movement disorder is tardive dyskinesia. In some embodiments, the hyperkinetic movement disorder is Tourette's syndrome. In some embodiments, the hyperkinetic movement disorder is Huntington disease (also called Huntington's disease). In some embodiments, the hyperkinetic movement disorder is tics. In some embodiments, the hyperkinetic movement disorder is chorea associated with Huntington disease (i.e., Huntington chorea, Huntington's chorea, or Huntington chronic progressive hereditary chorea). In some embodiments, the hyperkinetic movement disorder is ataxia, chorea, dystonia, Huntington disease, myoclonus, restless leg syndrome, or tremors.

In some embodiments, the pharmaceutical compositions of the present disclosure may be useful in preventing or reducing the risk of developing the disease; e.g., preventing or reducing the risk of developing a disease, condition or disorder in an individual who may be predisposed to the disease, condition or disorder but does not yet experience or display the pathology or symptomatology of the disease.

In some embodiments, the patient has 22q11.2 deletion syndrome. In some embodiments, the patient is predisposed to developing a psychiatric disorder due to the patient having 22q11.2 deletion syndrome.

In some embodiments, the patient has catechol-O-methyltransferase (“COMT”) haploinsufficiency. In some embodiments, the patient is predisposed to developing a psychiatric disorder due to the patient having COMT haploinsufficiency.

Hyperkinetic movement disorders represent a category of neurological disorders that are characterized by unwanted and uncontrollable, or poorly controllable, involuntary movements. The phenomenology of these disorders is quite variable encompassing chorea, tremor, dystonia, myoclonus, tics, other dyskinesias, jerks and shakes. Hyperkinetic movement disorders include ataxia, chorea, dystonia, hemifacial spasm, Huntington's disease, chorea associated with Huntington's disease, myoclonus, restless leg syndrome, tardive dyskinesia, tics, Tourette's syndrome, and tremors.

Mood disorders represent a category of mental disorders in which the underlying problem primarily affects a person's persistent emotional state (their mood). Mood disorders include: major depressive disorder (also called major depression), bipolar disorder, persistent depressive disorder (long lasting low grade depression), cyclothymia (a mild form of bipolar disorder), catatonic depression, post-partum depression, mania, and seasonal affective disorder (SAD). Mood disorders include substance-induced mood disorders and mood disorders due to a medical condition, e.g., hypothyroidism or Parkinson's disease.

Bipolar disorder, also known as bipolar affective disorder or manic-depressive illness, is a mental disorder characterized by periods of elevated mood and periods of depression. The periods of elevated mood is known as mania or hypomania depending on the severity or whether psychosis is present. Symptoms of mania or a manic episode include a long period of feeling “high” or an overly happy or outgoing mood, extreme irritability, talking very fast, racing thoughts, jumping from one idea to another, being easily distracted, increasing activities, being overly restless, sleeping little, having an unrealistic belief in one's abilities, impulsive behavior, and engaging in pleasurable, high-risk behaviors. Symptoms of depression or a depressive episode include: an overly long period of sadness or hopelessness, loss of interest in activities, feeling tired, problems with concentration or memory, difficulty making decisions, being restless or irritable, change in eating or sleeping habits, and suicide ideation. Patients with bipolar disorder have a high risk of suicide and self-harm.

Schizoaffective disorder is a mental health condition characterized primarily by symptoms of schizophrenia, such as hallucinations or delusions, and symptoms of a mood disorder, such as mania and depression.

Obsessive-compulsive disorder (OCD) is an anxiety disorder characterized by recurrent and persistent anxiety-provoking thoughts (obsessions) that lead to repetitive behaviors (compulsions) that focus on alleviating distress caused by obsessive thoughts. Patients may or may not recognize that the obsessions and compulsions are unreasonable, and these thoughts and behaviors can become time-consuming and impair function.

Lesch-Nyhan syndrome is characterized by neurologic dysfunction, cognitive and behavioral disturbances, and uric acid overproduction and has a prevalence of 1:380,000. Patients with this syndrome suffer from cognitive deficits, movement disorders, and self-injurious behavior. The most common presenting feature of Lesch-Nyhan syndrome is developmental delay during the first year of life; hypotonia and delayed motor skills are usually evident by age 3-6 months. Children with Lesch-Nyhan syndrome typically fail to sit, craw, and walk, and are ultimately confined to a wheelchair. Even with effective management of symptoms, most affected individuals survive only into their second or third decade.

Agitation in Alzheimer's disease refers to a cluster of several behavioral symptoms associated with the disease. Agitation develops as the disease progresses and occurs in addition to cognitive loss. The cluster of symptoms includes anxiety, depression, irritability, and motor restlessness (such as pacing, wandering, constant movement). Other symptoms that may occur include sleep disturbances, delusions, hallucinations, compulsive behaviors, aggression, and general emotional distress. Agitation may occur in as many as half of all individuals with Alzheimer's disease. Agitation is associated with patients who have a poor quality of life, deteriorating family relationships and professional caregivers, ultimately leading to admission to a residential care facility.

Fragile X syndrome (also called Martin-Bell syndrome) is a genetic condition that causes a range of developmental problems including learning disabilities and cognitive impairment. Usually, males are more severely affected by this disorder than females. Fragile X syndrome is inherited in an X-linked dominant pattern. Affected individuals usually have delayed development of speech and language by the age of 2 years. Most males with Fragile X syndrome have mild to moderate intellectual disability, while about one-third of affected females are intellectually disabled. Children with Fragile X syndrome may also exhibit behavioral problems, including anxiety, attentional deficits, anxiety, and hyperactive behaviors, such as fidgeting or impulsive actions.

Autism spectrum disorder (ASD) is a range of complex neurodevelopment disorders, characterized by social impairments; communication difficulties; and restricted, repetitive, and stereotyped patterns of behavior (stereotypies). Autistic disorder, sometimes called autism or classical ASD, is the most severe form of ASD. Other conditions include a milder form known as Asperger syndrome, childhood disintegrative disorder, pervasive developmental disorder, which is not otherwise specified (usually referred to as PDD-NOS). Although ASD varies significantly in character and severity, it occurs in all ethnic and socioeconomic groups and affects every age group.

Major depressive disorder is one of the most common mental illnesses. Depression causes people to lose pleasure from daily life, can complicate other medical conditions, and can even be serious enough to lead to suicide. Depression can occur to anyone, at any age, and to people of any race or ethnic group.

Rett syndrome (RTT), originally termed cerebroatrophic hyperammonemia, is a rare genetic postnatal neurological disorder of the grey matter of the brain that affects both females and male patients, with predominance of female ones. Rett syndrome causes problems in brain function that are responsible for cognitive, sensory, emotional, motor, and autonomic function. Most frequent problems that occur include those involving learning, speech, sensory sensations, mood, movement, breathing, cardiac function, chewing, swallowing, and digestion. It can be characterized by normal early growth and development followed by a slowing of development, loss of purposeful use of the hands, distinctive hand movements, slowed brain and head growth, problems with walking, seizures, and intellectual disability.

Chorea-acanthocytosis (ChAc) is a neurological disorder that affects movements in many parts of the body. Chorea refers to the involuntary jerking movements made by people with this disorder. People with this condition also have abnormal star-shaped red blood cells (acanthocytosis). This disorder is one of a group of conditions called neuroacanthocytoses that involve neurological problems and abnormal red blood cells.

As used herein, “22q11.2 Deletion Syndrome,” (22q11.2 DS), is also known as Velocardiofacial syndrome (“VCFS”), DiGeorge syndrome, or CATCH 22, and less often referred to as DiGeorge sequence, Microdeletion 22q11.2, Monosomy 22q1, Conotruncal anomaly face syndrome, Sedlačková syndrome, Shprintzen syndrome, Takao syndrome, or Cayler cardiofacial syndrome.

As used herein, “COMT” is a key enzyme for regulating catechol compounds, including dopamine, epinephrine and norepinephrine. Individuals with VCFS have approximately 50% less COMT mRNA, COMT protein expression, and enzyme activity compared to normal subjects. The characteristic behavioral manifestations of VCFS may be related to dopamine dysregulation resulting from COMT haploinsufficiency.

### EXAMPLES

The disclosure further provides specific examples. The following examples are offered for illustrative purposes, and are not intended to limit the disclosure in any manner. Those of skill in the art will readily recognize a variety of non-critical parameters which can be changed or modified to yield essentially the same results.

### Example 1. Preparation of Granules

### Materials

TABLE 1A

| | | | |
|---|---|---|---|
| | | Net | |
| | | Weight | % |
| Item # | Ingredients | (kg) | (w/w) |

PRE-BLEND

| | | | |
|---|---|---|---|
| 1 | Valbenazine Ditosylate | 3.640 | 40.0 |
| 2 | Silicified microcrystalline cellulose | 2.275 | 25.0 |
| | (PROSOLV ® 90) | | |
| 3 | Isomalt (GALENIQ ™ 720) | 1.820 | 20.0 |
| 4 | Partially pregelatinized maize starch | 0.683 | 7.5 |
| | (STARCH 1500 ®) | | |
| 5 | Hypromellose (METHOCEL ™ E5 Premium | 0.455 | 5.0 |
| | LV) | | |
| 6 | Magnesium stearate (Ligamed MF-2-V) | 0.136 | 1.5 |

FINAL BLEND

| | | | |
|---|---|---|---|
| 7 | Magnesium stearate (Ligamed MF-2-V) | 0.091 | 1.0 |
| | Total Weight | 9.084 | |

### Pre-Blend, 30-L Bin, Comil, and Fitzmil

To a 30-L bin blender was added the following ingredients in order: isomalt (GalenIQ™ 720, 1.820 kg, Item 3), valbenazine ditosylate (3.640 kg, Item 1), and silicified microcrystalline cellulose (PROSOLV® 90, 2.275 kg, Item 2). The lid to the bin was secured, and the ingredients were blended at 20 rpm for 4 minutes. The blended ingredients were transferred to a conical screen mill (COMIL®) configured with a 0.8 mm screen and 0.150-inch spacers at about 1500 rpm. The milled ingredients were added to the bin and the lid secured. The ingredients were blended for 25 minutes and 30 seconds with a blender rotation speed set to 20 rpm.

To a conical screen mill with a 0.8 mm screen and 0.150-inch spacers at about 1500 rpm were transferred the following ingredients in order, partially pregelatinized maize starch (STARCH 1500®, 0.683 kg, Item 4) and hypromellose (METHOCEL™ E5 Premium LV, 0.455 kg, Item 5). The milled material was transferred to the bin and the lid secured. The ingredients were blended with a blender rotation speed set to 20 rpm for 10 minutes and 15 seconds.

Magnesium stearate (Ligamed MF-2-V, 0.136 kg, Item 6) was screened by hand through an 18-mesh screen. The screened material was added to the 30-L bin and the lid secured. The ingredients were blended with a blender rotation speed set to 20 rpm for 3 minutes. After blending, the blended ingredients (i.e., Items 1-6) were discharged to a polyethylene-lined in-process container.

### Roller Compaction and Fitzmilling

A Fitzmill IR 220 Roller Compactor, affixed with a polyethylene-lined in-process container at the roller compactor discharge, was configured with the following operation parameters: 1) roll speed at 3.0 rpm, 2) VFS screw speed at 250 rpm, 3) HFS screw speed at 55 rpm, and 4) roll force at 2710 lb/inch. The roller compactor and hopper vibrator were started and the blended ingredients (i.e., Items 1-6) were loaded portion-wise to the roller compactor.

A Model M5A Fitzmill in knife configuration was equipped with a 20-mesh screen and set to 1200 rpm. The roller-compacted ribbon was slowly transferred through the Fitzmill to give 7.448 kg of the granulated material, which was placed into a polyethylene-lined in-process container.

### Final Blend, 30-L Bin Blender

To a 30-L Bin Blender was added the following ingredients in order: the milled ribbon material prepared as described above, and sieved magnesium stearate (Ligamed MF-2-V, adjusted to 1.0% based upon intra-granular yield, added 0.075 kg, Item 7). The lid to the bin was secured and the ingredients were blended at 20 rpm for 3 minutes. The final blend was discharged into a double polyethylene-lined container, containing two 4-unit desiccant bags between the two liners, to give 7.345 kg of the final blend.

### Press Batch Setup

A Fette 52i Press was configured with a 6 mm fill cam, dosing shims, and multi tip punches (2.2 mm). A polyethylene waste bag was placed under the tablet discharge chute for collection during setup. The press was set up with the following operation parameters: 1) turret speed at 35 rpm, 2) fill depth setting at 3.42 mm, 3) pre-compression setting at 1.83 mm, 4) pre-compression setting at 0.70 kN, 5) main compression setting at 1.66 mm, 6) main compression setting at 5.00 kN, 7) feeder 1 setting at 50 rpm, and 8) number of punches at 16 with 10 tips each. The Fette 52i Press hopper was filled with the final blend and the press started. After approximately 5 g of tablets were collected, the press was stopped, and the tablets were tested and the press adjusted as necessary. The target and target range for the granule(s) are as follows: weight for each granule, 9.12 mg, range of 8.21-10.03 mg; combined weight for 10 granules, 91.20 mg, range 86.64-95.76 mg; hardness, 1.6 kilopond (kp); friability, not more than (NMT) 1.0%; and appearance, no defects.

### Compression of Granules

Once the press was adjusted to provide the granule properties with the target ranges, the waste bag was removed, and a new polyethylene bag was placed under the tablet discharge chute to collect the acceptable granules. The press was started, and the granules were collected.

### Metal Check

The granules were passed through a metal detector. All of the granules that passed and were deemed acceptable were collected into a double polyethylene-lined container containing one 8-unit desiccant, to give 6.904 kg (excludes the weight of the in-process testing material, 0.109 kg); total weight was 7.013 kg (77% yield) for the finished granules (uncoated). The average weight for a granule was determined to be 9.195 mg (3.7 mg of valbenazine ditosylate or 2.0 mg of valbenazine free base), and the theoretical number of granules produced from the process was 750,843 (6.904 kg/9.195 mg×1,000,000).

### Example 2. Preparation of Coated Granules

### Materials

TABLE 2

| | | Net Weight | |
|---|---|---|---|
| Item # | Ingredients | (kg) | % (w/w) |
| 1 | Uncoated Granules | 6.904 | 90.91 |
| 2 | Purified Water USP | (10.312)^{A} | — |
| 3 | OPADRY ® II White (85F18422) | 1.820 ^{B} | 9.09 |
| | Total Weight | 8.724 | |

^{A}Item 2 is substantially removed during the process
^{B} Corresponds to a target 10.0% Weight Gain

### Preparation of Coating Solution

A stainless-steel vessel (NLT 20-L) was filled with 10.312 kg of purified water USP (Item #2). Moderate agitation was initiated to generate a vortex and OPADRY® II White (1.820 kg, Item #3) was slowly added. Once the addition of OPADRY® II White was complete, the contents were mixed for not less than 30 minutes and until a solution was obtained (34 minutes). The agitation was reduced such that the solution was still moving, and an appearance of a slight vortex was observed. The solution was used within 24 hours of preparation.

### Preparation of Spray System

A GPCG-5 Wurster Spray System was configured as follows: partition length, 18″; air distribution plates, D; product support screen, 100 mesh; partition height from distribution plate, 25+/−2 mm; nozzle tip port size, 1.0 mm; nozzle cap height, flush with nozzle tip; solution tubing size, MASTERFLEX® 14 mm; process, aqueous; filter bag shake Interval/Duration, 30 seconds/5 seconds. The nozzle was manually checked with purified water USP using the following set-points: 6 mL/min. for the spray rate and 1.0 bar for the atomization air pressure.

### Coating Process Parameters

TABLE 3

| Processing | Steady State Target | Range |
|---|---|---|
| Coating | | |
| Process Air Temperature (° C.) | 52 | 40-65 |
| Process Air Volume (cfm) | 135 | 80-200 |
| Spray Rate (mL/min) | 8-20 (ramps up) | 4-24 |
| Product Temperature (° C.} | 45 | 43-47 |
| Drying | | |
| Process Air Temperature (° C.) | 52 | 44-60 |
| Process Air Volume (cfm) | 135 | 80-200 |
| Product Temperature (° C.} | 45-47 | 40-50 |
| Dew Point (° C.) | 6 | 0-12 |
| Atomization Air Pressure (bar) | 1.0 | 0.9-1.1 |

It will be understood that the coating process parameters in Table 3 are representative and other parameters may also be used. For instance, during the coating process, the process air temperature (° C.) may range from 55° C. to 72° C. with no impact on the quality of product; the process air volume (cfm) may range from 600 cfm to 900 cfm with no impact on the quality of product; a spray rate up to 170 g/min may be used with no impact on the quality of product; deviations in product temperature above 48° C. may occur for up to 5-8 min (up to a maximum of 51-52° C.) with no impact on the product quality.

### Equipment Preparation and Granule Charging

The GPCG-5 Wurster Spray System was preconditioned to the following processing parameters: process air temperature at about 60° C.; process air volume at 140 cfm; and dew point at about 6° C. Once the product temperature reached 50° C. the GPCG-5 was shut down and uncoated granules of Example 1 (Item #1) were charged into the product chamber. The chamber back was inserted into the GPCG-5 unit, and then the spray transfer line, temperature probe, and atomization airline were attached to the nozzle inlet. The product bowl was compressed to preheat the granules, and the dryer blower was initiated.

### Coating

After fluidization was achieved, 40 granules were collected from the GPCG-5 sample port and the average weight determined (9.125 mg). Based on the average granule weight, the targeted 10% coat weight was calculated as 10.038 mg. The spray rate was initiated at 8 mL/min. and increased every 10 minutes by 4 mL/min until a spray rate of 20 mL/min was achieved. The product at the sample port was inspected for aggregation once the spray rate of 20 mL/min was achieved and every 15 minutes thereafter. The temperatures, airflows, and product bed temperature were adjusted as needed to fall within the target ranges. Once the target coating weight was achieved, the solution pump was stopped, reversed flow, and the pump was restarted to clear the line. Simultaneously, the granules were allowed to continue fluidization for additional drying time until the product temperature increased by 2° C. and the unit was stopped. The coated granules were discharged from the product bowl and sieved through a dedicated US Standard size 7 and 10 mesh sieves. The material that was <7 mesh and >10 mesh size was retained. No oversized (>7 mesh) or undersized (<10 mesh) material was identified. The coated granules were placed in a double polyethylene-line container with one 8-unit desiccant, 7.523 kg (weight includes 0.020 kg analytical sample), 99% yield.

### Encapsulation

Coated granules were filled into designed to be opened sprinkle capsules, i.e., CAPSUGEL® Size 0 CONI-SNAP® Sprinkle capsules, to achieve target dosage strengths of 20 mg, 40 mg, 60 mg, and 80 mg valbenazine utilizing an IMA Adapta encapsulator, configured with a pellet dosator configured with Size 00 pellet doser and pistons from the Size 0 pellet doser. Granule dosing was controlled by volume to achieve the 80 mg strength. Capsules were also filled manually, by count, to achieve lower strengths.

Additional batches (utilizing equivalent upstream processes) utilized an IMA Adapta encapsulator configured with a counting wheel and vacuum. Dosing was controlled by count, utilizing a dosator and wheel bored with holes to provide automated encapsulation and better control over unit dosing.

Accordingly, the coated granules have a composition as shown in Table 1B:

TABLE 1B

| | | |
|---|---|---|
| Item | | % (w/w) |
| # | Ingredients | formulation |

Granules

| | | |
|---|---|---|
| 1 | Valbenazine ditosylate | 36.36 |
| 2 | Silicified microcrystalline cellulose | 22.73 |
| | (PROSOLV ® 90) | |
| 3 | Isomalt (GALENIQ ™ 720) | 18.18 |
| 4 | Partially pregelatinized maize starch | 6.82 |
| | (STARCH 1500 ®) | |
| 5 | Hypromellose (METHOCEL ™ E5 Premium LV) | 4.55 |
| 6 | Magnesium stearate (Ligamed MF-2-V) | 2.27 |

Coating

| | | |
|---|---|---|
| 7 | OPADRY ® II White (85F18422) | 9.09 |
| | Total | 100 |

### Example 3. Dissolution Profile of Coated Granules

### Materials

TABLE 4
Chemicals
Valbenazine ditosylate reference standard (stored below 30° C.)
Concentrated Hydrochloric Acid (HCl, 12N), ACS Grade or equivalent
Ortho-Phosphoric Acid (85%) HPLC Grade or equivalent
Acetonitrile, HPLC grade
Methanol, HPLC grade
Purified Water, USP grade
Purified pepsin, not less than 10,000 activity units/mg (VWR, Cat#
0717-100G or equiv.)
Equipment
In-line full flow filters, 1 μm (UHMW PE)
Suitable HPLC system equipped with UV/Vis detector
HPLC Column- PHENOMENEX ® Kinetex C18 4.6 × 100 mm, 2.6 μm
(or equivalent)
Analytical balance with a precision of at least 0.01 mg
Suitable Dissolution Apparatus with USP 1 (Baskets)
Suitable Dissolution Apparatus with USP 2 (Paddles)
Class A volumetric glassware
Sonicator
Wire helix sinker (QLA Part# CAPWHT-2S)

### General Methods

Dissolution standards and sample preparations described below were analyzed by HPLC. Instrument parameters were as described in Table 5, below:

TABLE 5

| | |
|---|---|
| Column | PHENOMENEX ® Kinetex C18, 4.6 × 100 mm, |
| | 2.6 μm |
| Mobile phase A | 0.05% Phosphoric Acid in Water |
| Mobile phase B | Acetonitrile (100%) |
| Flow | 1.0 mL/min |
| Wavelength | 280 nm; Band Width- 4.0 nm |
| Injection Volume | 10 μL |
| Column Temp. | 40° C. |
| Autosampler Temp. | Ambient |
| Needle Wash | H₂O/Methanol- 90/10 (v/v) |
| Run Time | 8.0 min; Valbenazine (approximately 4.0 min) |

The mobile phase gradient was as described in Table 6, below:

TABLE 6

| Time (min) | % A | % B |
|---|---|---|
| 0.0 | 95 | 5 |
| 0.5 | 95 | 5 |
| 5.0 | 55 | 45 |
| 5.2 | 95 | 5 |
| 8.0 | 95 | 5 |

The column was equilibrated at initial conditions before each sample sequence, and was washed after each sample sequence using a gradient of acetonitrile and H₂O/acetonitrile (90/10 (v/v)) flowing at 1.0 mL/min, at a column temperature of 50° C.

For each sample preparation described below, at each time point noted below, a % valbenazine (free base) release value was calculated as follows:

$$\% release = ( \frac{A s}{A s t d} \times Cstd \times \frac{P}{100} \times 0.5486 \times \frac{900}{1} ) \times \frac{100}{L C}$$

where
As=valbenazine area in Sample
Astd=average area of valbenazine peak of dissolution standard
Cstd=concentration of valbenazine ditosylate in dissolution standard (mg/mL)
P=purity of valbenazine ditosylate reference standard
LC=label claim of valbenazine (20 mg, 40 mg, or 80 mg).

### Preparation of Dissolution Standards for Valbenazine

### 80-mg Dose

40.5±4.0 mg of valbenazine ditosylate reference standard (equivalent to 22.22 mg of valbenazine free base) was added to a 250-mL volumetric flask. Diluent (0.1 N HCl dissolution media) was added to the flask, up to about 75% of the volume of the flask. The contents of the flask were mixed well, and sonicated if necessary, to provide a standard solution containing 0.0889 mg/mL valbenazine free base. The standard was stable for 17 days, stored at ambient lab conditions.

### 20- and 40-mg Dose

10.12±1.0 mg of valbenazine ditosylate reference standard (equivalent to 5.55 mg of valbenazine free base) was added to a 250-mL volumetric flask. Diluent (0.1N HCl dissolution media) was added to the flask, up to about 75% of the volume of the flask. The contents of the flask were mixed well, and sonicated if necessary, to provide a standard solution containing 0.0222 mg/mL valbenazine free base. The standard was stable for 7 days, stored at ambient lab conditions.

### Dissolution Testing

### Testing Criteria

TABLE 7

| | Number | |
|---|---|---|
| | of units | |
| Stage | tested | Acceptance Criteria |
| S₁ | 6 | Each unit is NLT Q + 5% |
| S₂ | 6 | The average of 12 units (S₁ + S₂) is ≥Q, and no unit |
| | | is <Q − 15% |
| S₃ | 12 | The average of 24 units (S₁ + S₂ + S₃) is ≥Q, |
| | | NMT 2 units are <Q − 15%, and no unit is <Q − 25% |

At Stage S₁, 6 units were tested in Tier I media (0.1 N HCl).

If Stage S₁ criteria were not met, but there was no evidence of cross-linking (no pellicle was observed), Stage S₂ testing was conducted for 6 additional units in Tier I media, and results from the 12 units tested in Tier I media were assessed and reported. If Stage S₂ criteria were not met, Stage S₃ testing was conducted for 12 additional units in Tier I media, and results from the 24 units tested in Tier I media were assessed and reported.

In Stage S₁ criteria were not met and there was evidence of cross-linking (pellicle observed), Stage S₁ testing was repeated in Tier II media (0.1N HCl with pepsin, 700,000-750,000 activity units per liter of media). If Stage S₁ criteria were not met in Tier II media, Stage S₂ testing was conducted for 6 additional units in Tier II media, and results from the 12 units tested in Tier II media were assessed and reported. If Stage S₂ criteria were not met, Stage S₃ testing was conducted for 12 additional units in Tier II media, and results from the 24 units tested in Tier II media were assessed and reported.

### Testing Parameters

80-, 40-, and 20-mg doses of coated oral granules prepared according to Example 2 were removed from the capsule and tested according to Table 8, below:

TABLE 8

| | |
|---|---|
| Type | USP 1 Apparatus- Baskets |
| Rotation Speed | 50 rpm |
| Bath Temperature | 37.0 ± 0.5° C. |
| Dissolution Media | Tier 1: 0.1N HCl (Degassed before use) |
| Dissolution Volume | 900 mL |
| Time Points for HPLC | 5, 10, 15, 20, 30 and 60 minutes |
| Analysis | |
| HPLC Sample Volume | 1.2 mL |
| Filter Type | 1 μm In-line full flow filter (UHMW PE) |

80-, 40-, and 20-mg doses of coated oral granules prepared according to Example 2 were tested as whole capsules according to Table 9, below:

TABLE 9

| | |
|---|---|
| Type | USP 2 Apparatus- Paddles |
| Rotation Speed | 50 rpm |
| Bath Temperature | 37.0 ± 0.5° C. |
| Dissolution Media: | Tier I: 0.1N HCl (Degassed before use) |
| | Tier II: 0.1N HCl with pepsin |
| Dissolution Volume | 900 mL |
| Time Points for HPLC Analysis | 5, 10, 15, 20, 30 and 60 minutes |
| HPLC Sample Volume | 1.2 mL |
| Sinker Type | Wire helix sinker |

### Testing Procedure—80-Mg Dose, Capsules Removed

Each dissolution vessel was filled with 900 mL of degassed dissolution media. The temperature of media for each vessel was recorded prior to starting the dissolution run.

Six valbenazine sprinkle capsules were weighed, and the weight recorded. The contents of each capsule was carefully removed and transferred into the respective baskets. The weight of each empty capsule shell was recorded.

Dissolution was measured according to Table 8, above. After completion of the dissolution, the end temperature and observations for each vessel were recorded. The dissolution sample was stable for 12 days, stored at ambient lab conditions.

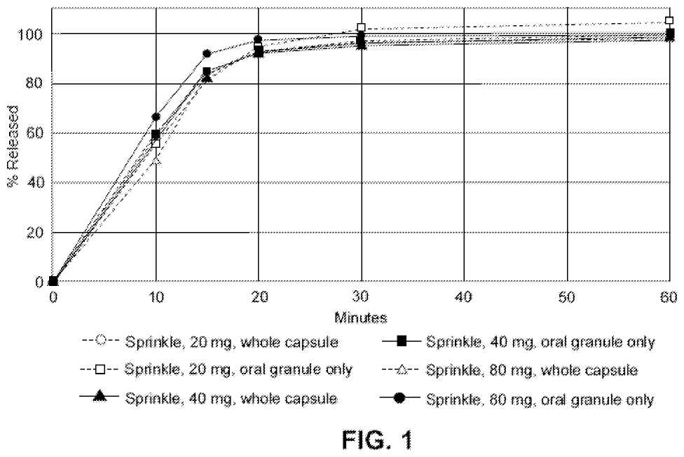

Dissolution in Tier I media, shown in FIG. 1, satisfied the Stage S₁ acceptance criteria.

### Testing Procedure—40- and 20-Mg Dose, Capsules Removed

Each dissolution vessel was filled with 900 mL of degassed dissolution media. The temperature of media for each vessel was recorded prior to starting the dissolution run.

Six valbenazine sprinkle capsules were weighed, and the weight recorded. The contents of each capsule was carefully removed and transferred into the respective baskets. The weight of each empty capsule shell was recorded.

Dissolution was measured according to Table 8, above. After completion of the dissolution, the end temperature and observations for each vessel were recorded. The dissolution sample was stable for 7 days, stored at ambient lab conditions.

Dissolution in Tier I media, shown in FIG. 1, satisfied the Stage S₁ acceptance criteria.

### Testing Procedure—80-Mg Dose, Whole Capsule

Each dissolution vessel was filled with 900 mL of degassed dissolution media. The temperature of media for each vessel was recorded prior to starting the dissolution run. Six valbenazine sprinkle capsules were weighed, and the weight recorded. Each capsule was fitted with a wire helix sinker and introduced into the respective vessels.

Dissolution was measured according to Table 9, above. After completion of the dissolution, the end temperature and observations for each vessel were recorded. The dissolution sample was stable in Tier I media for 12 days, stored at ambient lab conditions. The dissolution sample was stable in Tier II media for 7 days, stored at ambient lab conditions.

Dissolution in Tier II media, shown in FIG. 1, satisfied the Stage S₁ acceptance criteria.

### Testing Procedure—40- and 20-Mg Dose, Whole Capsule

Each dissolution vessel was filled with 900 mL of degassed dissolution media. The temperature of media for each vessel was recorded prior to starting the dissolution run. Six valbenazine sprinkle capsules were weighed, and the weight recorded. Each capsule was fitted with a wire helix sinker and introduced into the respective vessels.

Dissolution was measured according to Table 9, above. After completion of the dissolution, the end temperature and observations for each vessel were recorded. The dissolution sample was stable in Tier I media for 7 days, stored at ambient lab conditions. The dissolution sample was stable in Tier II media for 7 days, stored at ambient lab conditions.

Dissolution in Tier II media, shown in FIG. 1, satisfied the Stage S₁ acceptance criteria.

### Example 4. Oral Granules for Sprinkle Capsules G-Tube Suitability Evaluation

The purpose of this study was to determine the suitability of valbenazine oral granules for sprinkle capsule for administration by gastronomy tube (G-tube) for clinical use. The doses for this study were 20 mg, 40 mg, 60 mg, and 80 mg.

### Selection of Gastronomy Tubes

Silicone material was selected since it is a commonly used gastronomy tube material and a size range of 12 Fr to 18 Fr was selected based on the needs of the patient population. A gastronomy tube length of 8 inches was used throughout the study.

### Dispersion Media and Dispersion Preparation

Water was selected as the dispersion media for ease and availability for the caregivers/patients. Water is easily accessible and accommodates all diets. Warm water (warm to touch) is required for administration of this product through the gastronomy tube. The warm water ensures an adequate dose is delivered and prevents gastronomy tube clogging since the warm water dissolves the product in the dispersion media rather than suspends the product.

Caregivers/patients typically administer medication through gastronomy tubes by mixing the drug with the dispersion media and then drawing it up in a syringe and pushing through the gastronomy tube. Then, a flush is performed with the dispersion media to remove any remaining material from the gastronomy tube.

### Gastronomy Tube Suitability Evaluation:

The suitability evaluation was conducted by performing recovery testing. Recovery testing was carried out by administration through the G-tube and determining the % recovery at the lowest and highest dose. Acceptance criteria was set to % recovery of a 90.0-110.0%.

Soak time (30 minutes and 45 minutes) with ambient tap water was evaluated first for the bracketing gastronomy tube size diameters of 12 Fr and 18 Fr, and the bracketing doses of 20 mg and 80 mg. Since the 80 mg testing failed the ambient tap water soak time of 30 minutes and 45 minutes, it as decided to move forward with warm tap water (warm to the touch). It is possible that ambient tap water could be used with a soak time duration of more than 45 minutes. However, this path was not further explored due to the inconvenience it would cause the caregiver/patient with the extended preparation time.

Table 10 shows results from the % recovery testing.

TABLE 10
Gastronomy Tube Suitability Evaluation in Lukewarm Water (Approx. 30° C.-40° C.)

| | | | | | Rinse | | | | | |
|---|---|---|---|---|---|---|---|---|---|---|
| | | | | | Volume | | | | | |
| | | Volume of | | No. of | (Cup) then | | Extra | | | |
| | G-tube | Dispersing | Soaking | Cup | through | | G-tube | | Individual | Overall |
| Dose | Diameter | Media | Time | Rinses | G-tube | | rinse | Result | Pass/Fail | Pass/Fail |
| 20 mg | 12FR | 10 mL | 20 | 1 | 10 | mL | N/A | 96.0% | Pass | Pass |
| | | (30° C.) | minutes | | | | | | | |
| | | 10 mL | | | | | | 96.8% | Pass | |
| | | (30° C.) | | | | | | | | |
| | | 10 mL | | | | | | 97.6% | Pass | |
| | | (30° C.) | | | | | | | | |
| | | 10 mL | 15 | | | | | 97.9% | Pass | |
| | | (35° C.) | minutes | | | | | | | |
| | | 10 mL | | | | | | 96.7% | Pass | |
| | | (34° C.) | | | | | | | | |
| | | 10 mL | | | | | | 95.4% | Pass | |
| | | (32° C.) | | | | | | | | |
| 80 mg | | 10 mL | 15 | 1 | 10 | mL | | 96.7% | Pass | Fail |
| | | (40° C.) | minutes | | | | | | | |
| | | 10 mL | | | | | | 94.5% | Pass | |
| | | (34° C.) | | | | | | | | |
| | | 10 mL | | | | | | G-tube | Fail | |
| | | (38° C.) | | | | | | clogged | | |
| | | 10 mL | 20 | | | | | 93.9% | Pass | |
| | | (27° C.) | minutes | | | | | | | |
| | | 10 mL | | | | | | 89.7% | Fail | |
| | | (28° C.) | | | | | | | | |
| | | 10 mL | | | | | | 97.5% | Pass | |
| | | (28° C.) | | | | | | | | |
| | | 10 mL | 20 | 1 | 5 | mL | 5 mL | 96.4% | Pass | Pass |
| | | (35° C.) | minutes | | | | | | | |
| | | 10 mL | | | | | | 95.7% | Pass | |
| | | (34° C.) | | | | | | | | |
| | | 10 mL | | | | | | 90.8% | Pass | |
| | | (33° C.) | | | | | | | | |

Evaluation of the first administration (10 mL) with warm water and without rinsing the cup was performed to determine if rinsing of the cup was necessary to deliver the intended dose. The evaluation was performed using the 12 Fr gastronomy tube and bracketing doses of 20 mg and 80 mg. The evaluation determined that rinsing the cup with 5 mL after administration is necessary to achieve adequate recovery.

### Example 5. Stability Testing

Dosage forms were stored at 25° C./60% relative humidity (RH), 30° C./75% RH and 40° C./75% RH for three months. 60 mg powder filled capsules were compared with 60 mg oral granules in sprinkle capsules. The results from stability testing are shown in Table 11. Under the test conditions, the oral granules described herein have acceptable stability at 3 months.

TABLE 11

| 60 mg powder filled | | 60 mg oral granules | |
|---|---|---|---|
| capsules (reference) | | in sprinkle capsules | |
| Start | 3 months | Start | 3 months |

25° C./60% RH

| | | | | |
|---|---|---|---|---|
| Valbenazine assay | 100.1 | 100.5 | 98.2 | 97.7 |
| Degradation products | | | | |
| Compound 1 | <0.05 | <0.05 | <0.05 | <0.05 |
| Compound 2 | <0.05 | <0.05 | <0.05 | <0.05 |
| Unspecified degradation | <0.05 | <0.05 | <0.05 | 0.05 |
| product(s), each | | | | |
| Total degradation products | <0.1 | <0.1 | <0.1 | 0.1 |

30° C./75% RH

| | | | | |
|---|---|---|---|---|
| Valbenazine assay | 100.1 | 99.9 | 98.2 | 97.8 |
| Degradation products | | | | |
| Compound 1 | <0.05 | <0.05 | <0.05 | <0.05 |
| Compound 2 | <0.05 | <0.05 | <0.05 | <0.05 |
| Unspecified degradation | <0.05 | <0.05 | <0.05 | 0.05 |
| product(s), each | | | | |
| Total degradation products | <0.1 | <0.1 | <0.1 | 0.1 |

40° C./75% RH

| | | | | |
|---|---|---|---|---|
| Valbenazine assay | 100.1 | 99.5 | 98.2 | 97.3 |
| Degradation products | | | | |
| Compound 1 | <0.05 | <0.05 | <0.05 | <0.05 |
| Compound 2 | <0.05 | <0.05 | <0.05 | 0.08 |
| Unspecified degradation | <0.05 | 0.06 | <0.05 | 0.05 |
| product(s), each | | | | |
| Total degradation products | <0.1 | 0.1 | <0.1 | 0.1 |

Various modifications, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A pharmaceutical dosage form comprising a plurality of granules, wherein each granule comprises:
(a) an average diameter of at least 1 mm,
(b) at least one pharmaceutically acceptable carrier;
(c) an amount of valbenazine, or a pharmaceutically acceptable salt thereof; and
(d) a film-coating.

2. The pharmaceutical dosage form of claim 1, wherein each granule has an average diameter of about 1.5 mm to about 5 mm.

3. The pharmaceutical dosage form of claim 1, wherein each granule has an average diameter of about 2 mm to about 3 mm.

4. The pharmaceutical dosage form of claim 1, wherein each granule has an average diameter of about 2.15 mm to about 2.25 mm.

5. The pharmaceutical dosage form of claim 1, wherein each granule has an average diameter of about 2.18 mm to about 2.23 mm.

6. The pharmaceutical dosage form of claim 1, wherein each granule an average diameter of about 2.19 mm to about 2.21 mm.

7. The pharmaceutical dosage form of claim 1, wherein each granule has an average diameter of about 2.2 mm.

8. The pharmaceutical dosage form of any one of claims 1-7, wherein each granule has a diameter variation of no more than 20% from the average diameter.

9. The pharmaceutical dosage form of any one of claims 1-7, wherein each granule has a diameter variation of no more than 10% from the average diameter.

10. The pharmaceutical dosage form of any one of claims 1-9, wherein each granule has a d99 particle diameter distribution of at most about 2.8 mm.

11. The pharmaceutical dosage form of any one of claims 1-9, wherein each granule has a d99 particle diameter distribution of at most about 2.5 mm.

12. The pharmaceutical dosage form of any one of claims 1 to 11, wherein each granule has an average density of at least about 0.5 g/cm³.

13. The pharmaceutical dosage form of any one of claims 1 to 11, wherein each granule has an average density of at least about 1 g/cm³.

14. The pharmaceutical dosage form of any one of claims 1 to 11, wherein each granule has an average density of about 1 g/cm³ to about 2 g/cm³.

15. The pharmaceutical dosage form of claim 1, wherein each granule has:
(a) an average diameter of at most about 2.5 mm;
(b) a d99 particle diameter distribution of at most about 2.8 mm; and
(c) an average density of about 0.75 g/cm³ to about 2.5 g/cm³.

16. The pharmaceutical dosage form of any one of claims 1 to 15, wherein each granule has an average hardness of about 0.5 kp to about 3 kp.

17. The pharmaceutical dosage form of any one of claims 1 to 15, wherein each granule has an average hardness of about 0.8 kp to about 2.6 kp.

18. The pharmaceutical dosage form of any one of claims 1 to 15, wherein each granule has an average hardness of about 1 kp to about 2.4 kp.

19. The pharmaceutical dosage form of any one of claims 1 to 15, wherein each granule has an average hardness of about 1.2 kp to about 2 kp.

20. The pharmaceutical dosage form of any one of claims 1 to 15, wherein each granule has an average hardness of about 1.4 kp to about 1.8 kp.

21. The pharmaceutical dosage form of any one of claims 1 to 15, wherein each granule has an average hardness of about 1.5 kp to about 1.7 kp.

22. The pharmaceutical dosage form of any one of claims 1 to 15, wherein each granule has an average hardness of about 1.6 kp.

23. The pharmaceutical dosage form of any one of claims 1 to 22, wherein each granule has an average weight of about 8 mg to about 10.2 mg.

24. The pharmaceutical dosage form of any one of claims 1 to 22, wherein each granule has an average weight of about 8.2 mg to about 10.1 mg.

25. The pharmaceutical dosage form of any one of claims 1 to 22, wherein each granule has an average weight of about 8.3 mg to about 9.9 mg.

26. The pharmaceutical dosage form of any one of claims 1 to 22, wherein each granule has an average weight of about 8.5 mg to about 9.7 mg.

27. The pharmaceutical dosage form of any one of claims 1 to 22, wherein each granule has an average weight of about 8.7 mg to about 9.5 mg.

28. The pharmaceutical dosage form of any one of claims 1 to 22, wherein each granule has an average weight of about 8.9 mg to about 9.3 mg.

29. The pharmaceutical dosage form of any one of claims 1 to 22, wherein each granule has an average weight of about 9 mg to about 9.2 mg.

30. The pharmaceutical dosage form of any one of claims 1 to 22, wherein each granule has an average weight of about 9.1 mg.

31. The pharmaceutical dosage form of any one of claims 1 to 30, wherein each granule is stable to a film-coating process.

32. The pharmaceutical dosage form of any one of claims 1 to 31, wherein each granule is suitable for oral administration.

33. The pharmaceutical dosage form of any one of claims 1 to 32, wherein the film-coating comprises a film-forming polymer.

34. The pharmaceutical dosage form of claim 33, wherein the film-forming polymer is poly(vinyl alcohol).

35. The pharmaceutical dosage form of any one of claims 1 to 34, wherein the film-coating comprises a film-forming polymer, and the film-forming polymer is about 25 wt % to about 55 wt % of the weight of the film-coating.

36. The pharmaceutical dosage form of any one of claims 1 to 35, wherein the film-coating comprises a plasticizer.

37. The pharmaceutical dosage form of claim 36, wherein the plasticizer is polyethylene glycol, glycerin, or a mixture thereof.

38. The pharmaceutical dosage form of claim 36, wherein the plasticizer is polyethylene glycol.

39. The pharmaceutical dosage form of any one of claims 1 to 38, wherein the film-coating comprises a plasticizer, and the plasticizer is about 5 wt % to about 30 wt % of the weight of the film-coating.

40. The pharmaceutical dosage form of any one of claims 1 to 39, wherein the film-coating comprises a filler.

41. The pharmaceutical dosage form of claim 40, wherein the filler is talc.

42. The pharmaceutical dosage form of any one of claims 1 to 41, wherein the film-coating comprises a filler, and the filler is about 5 wt % to about 45 wt % of the weight of the film-coating.

43. The pharmaceutical dosage form of any one of claims 1 to 42, wherein the film-coating comprises a pigment/opacifier.

44. The pharmaceutical dosage form of claim 43, wherein the pigment/opacifier is titanium dioxide.

45. The pharmaceutical dosage form of any one of claims 1 to 44, wherein the film-coating comprises a pigment/opacifier, and the pigment/opacifier is up to about 40 wt % of the weight of the film-coating.

46. The pharmaceutical dosage form of any one of claims 1 to 45, wherein the film-coating comprises:
(a) about 25 wt % to about 55 wt % of a film-forming polymer;
(b) about 5 wt % to about 30 wt % of a plasticizer;
(c) about 5 wt % to about 45 wt % of a filler; and
(d) up to about 40 wt % of a pigment/opacifier; based on the weight of the film-coating.

47. The pharmaceutical dosage form of any one of claims 1 to 45, wherein the film-coating comprises:
(a) about 25 wt % to about 55 wt % of poly(vinyl alcohol);
(b) about 5 wt % to about 30 wt % of polyethylene glycol;
(c) about 5 wt % to about 45 wt % of talc; and
(d) up to about 40 wt % of titanium dioxide; based on the weight of the film-coating.

48. The pharmaceutical dosage form of any one of claims 1 to 32, wherein the film-coating comprises OPADRY® II.

49. The pharmaceutical dosage form of any one of claims 1 to 32, wherein the film-coating is OPADRY® II.

50. The pharmaceutical dosage form of any one of claims 1 to 49, wherein the film-coating makes up about 3.5 wt % to about 15 wt % of the weight of each granule.

51. The pharmaceutical dosage form of any one of claims 1 to 50, wherein the at least one pharmaceutically acceptable carrier comprises a diluent.

52. The pharmaceutical dosage form of claim 51, wherein the diluent is silicified microcrystalline cellulose, isomalt, or a mixture thereof.

53. The pharmaceutical dosage form of claim 51, wherein the diluent is a mixture of silicified microcrystalline cellulose and isomalt.

54. The pharmaceutical dosage form of any one of claims 51 to 53, wherein each granule comprises about 25 wt % to about 65 wt % of the diluent.

55. The pharmaceutical dosage form of any one of claims 51 to 53, wherein each granule comprises about 35 wt % to about 55 wt % of the diluent.

56. The pharmaceutical dosage form of any one of claims 51 to 53, wherein each granule comprises about 40 wt % to about 50 wt % of the diluent.

57. The pharmaceutical dosage form of any one of claims 51 to 53, wherein each granule comprises about 45 wt % of the diluent.

58. The pharmaceutical dosage form of any one of claims 1 to 57, wherein the at least one pharmaceutically acceptable carrier comprises a disintegrant.

59. The pharmaceutical dosage form of claim 58, wherein the disintegrant is partially pregeletanized maize starch.

60. The pharmaceutical dosage form of claim 58 or 59, wherein each granule comprises about 2 wt % to about 12 wt % of the disintegrant.

61. The pharmaceutical dosage form of claim 58 or 59, wherein each granule comprises about 5 wt % to about 10 wt % of the disintegrant.

62. The pharmaceutical dosage form of claim 58 or 59, wherein each granule comprises about 6 wt % to about 9 wt % of the disintegrant.

63. The pharmaceutical dosage form of claim 58 or 59, wherein each granule comprises about 7 wt % to about 8 wt % of the disintegrant.

64. The pharmaceutical dosage form of claim 58 or 59, wherein each granule comprises about 7.5 wt % of the disintegrant.

65. The pharmaceutical dosage form of any one of claims 1 to 64, wherein the at least one pharmaceutically acceptable carrier comprises a binder.

66. The pharmaceutical dosage form of claim 65, wherein the binder is hydroxypropyl methylcellulose.

67. The pharmaceutical dosage form of claim 65 or 66, wherein each granule comprises about 0.5 wt % to about 10 wt % of the binder.

68. The pharmaceutical dosage form of claim 65 or 66, wherein each granule comprises about 2 wt % to about 8 wt % of the binder.

69. The pharmaceutical dosage form of claim 65 or 66, wherein each granule comprises about 3 wt % to about 7 wt % of the binder.

70. The pharmaceutical dosage form of claim 65 or 66, wherein each granule comprises about 4 wt % to about 6 wt % of the binder.

71. The pharmaceutical dosage form of claim 65 or 66, wherein each granule comprises about 4.5 wt % to about 5.5 wt % of the binder.

72. The pharmaceutical dosage form of claim 65 or 66, wherein each granule comprises about 5 wt % of the binder.

73. The pharmaceutical dosage form of any one of claims 1 to 72, wherein the at least one pharmaceutically acceptable carrier comprises a lubricant.

74. The pharmaceutical dosage form of claim 73, wherein the lubricant is magnesium stearate.

75. The pharmaceutical dosage form of claim 73 or 74, wherein each granule comprises about 0.5 wt % to about 5 wt % of the lubricant.

76. The pharmaceutical dosage form of claim 73 or 74, wherein each granule comprises about 1 wt % to about 3 wt % of the lubricant.

77. The pharmaceutical dosage form of claim 73 or 74, wherein each granule comprises about 2 wt % to about 2.8 wt % of the lubricant.

78. The pharmaceutical dosage form of claim 73 or 74, wherein each granule comprises about 2.2 wt % to about 2.6 wt % of the lubricant.

79. The pharmaceutical dosage form of claim 73 or 74, wherein each granule comprises about 2.3 wt % to about 2.5 wt % of the lubricant.

80. The pharmaceutical dosage form of claim 73 or 74, wherein each granule comprises about 2.4 wt % to about 2.4 wt % of the lubricant.

81. The pharmaceutical dosage form of claim 73 or 74, wherein each granule comprises about 2.5 wt % of the lubricant.

82. The pharmaceutical dosage form of any one of claims 1 to 81, wherein each granule comprises about 5 wt % to about 40 wt % of valbenazine, or a pharmaceutically acceptable salt thereof, based on the weight of the free base.

83. The pharmaceutical dosage form of any one of claims 1 to 81, wherein each granule comprises about 10 wt % to about 40 wt % of valbenazine, or a pharmaceutically acceptable salt thereof, based on the weight of the free base.

84. The pharmaceutical dosage form of any one of claims 1 to 81, wherein each granule comprises about 15 wt % to about 30 wt % of valbenazine, or a pharmaceutically acceptable salt thereof, based on the weight of the free base.

85. The pharmaceutical dosage form of any one of claims 1 to 81, wherein each granule comprises about 19 wt % to about 25 wt % of valbenazine, or a pharmaceutically acceptable salt thereof, based on the weight of the free base.

86. The pharmaceutical dosage form of any one of claims 1 to 81, wherein each granule comprises about 20 wt % to about 24 wt % of valbenazine, or a pharmaceutically acceptable salt thereof, based on the weight of the free base.

87. The pharmaceutical dosage form of any one of claims 1 to 81, wherein each granule comprises about 21 wt % to about 23 wt % of valbenazine, or a pharmaceutically acceptable salt thereof, based on the weight of the free base.

88. The pharmaceutical dosage form of any one of claims 1 to 81, wherein each granule comprises about 21.5 wt % to about 22.5 wt % of valbenazine, or a pharmaceutically acceptable salt thereof, based on the weight of the free base.

89. The pharmaceutical dosage form of any one of claims 1 to 81, wherein each granule comprises about 21.9 wt % to about 22.1 wt % of valbenazine, or a pharmaceutically acceptable salt thereof, based on the weight of the free base.

90. The pharmaceutical dosage form of any one of claims 1 to 81, wherein each granule comprises about 22 wt % of valbenazine, or a pharmaceutically acceptable salt thereof, based on the weight of the free base.

91. The pharmaceutical dosage form of any one of claims 1 to 90, wherein valbenazine, or a pharmaceutically acceptable salt thereof, is valbenazine ditosylate.

92. The pharmaceutical dosage form of any one of claims 1 to 91, wherein each granule comprises:
(a) about 5 wt % to about 40 wt % valbenazine, or a pharmaceutically acceptable salt thereof, based on the weight of the free base;
(b) about 25 wt % to about 65 wt % of a diluent;
(c) about 0.5 wt % to about 15 wt % of a disintegrant;
(d) about 0.5 wt % to about 10 wt % of a binder; and
(e) about 0.5 wt % to about 5 wt % of a lubricant.

93. The pharmaceutical dosage form of any one of claims 1 to 91, wherein each granule comprises:
(a) about 30 wt % to about 50 wt % valbenazine ditosylate;
(b) about 25 wt % to about 65 wt % of a diluent;
(c) about 0.5 wt % to about 15 wt % of a disintegrant;
(d) about 0.5 wt % to about 10 wt % of a binder; and
(e) about 0.5 wt % to about 5 wt % of a lubricant.

94. The pharmaceutical dosage form of any one of claims 1 to 91, wherein each granule comprises:
(a) about 30 wt % to about 50 wt % valbenazine ditosylate;
(b) about 15 wt % to about 35 wt % silicified microcrystalline cellulose;
(c) about 10 wt % to about 30 wt % isomalt;
(d) about 0.5 wt % to about 15 wt % partially pregeletanized maize starch;
(e) about 0.5 wt % to about 10 wt % hydroxypropyl methylcellulose; and
(f) about 0.5 wt % to about 5 wt % of magnesium stearate.

95. The pharmaceutical dosage form of any one of claims 1 to 91, wherein each granule comprises:
(a) about 5 wt % to about 40 wt % valbenazine, or a pharmaceutically acceptable salt thereof, based on the weight of the free base;
(b) about 25 wt % to about 65 wt % of a diluent;
(c) about 0.5 wt % to about 15 wt % of a disintegrant;
(d) about 0.5 wt % to about 10 wt % of a binder;
(e) about 0.5 wt % to about 5 wt % of a lubricant; and
(f) about 3.5 wt % to about 15 wt % of the film-coating.

96. The pharmaceutical dosage form of any one of claims 1 to 91, wherein each granule comprises:
(a) about 30 wt % to about 50 wt % valbenazine ditosylate;
(b) about 25 wt % to about 65 wt % of a diluent;
(c) about 0.5 wt % to about 15 wt % of a disintegrant;
(d) about 0.5 wt % to about 10 wt % of a binder;
(d) about 0.5 wt % to about 5 wt % of a lubricant; and
(e) about 3.5 wt % to about 15 wt % of a film-coating comprising:
a film-forming polymer;
a plasticizer; and
a filler.

97. The pharmaceutical dosage form of any one of claims 1 to 91, wherein each granule comprises:
(a) about 30 wt % to about 50 wt % valbenazine ditosylate;
(b) about 15 wt % to about 35 wt % silicified microcrystalline cellulose;
(c) about 10 wt % to about 30 wt % isomalt;
(d) about 0.5 wt % to about 15 wt % partially pregeletanized maize starch;
(e) about 0.5 wt % to about 10 wt % hydroxypropyl methylcellulose; and
(f) about 0.5 wt % to about 5 wt % magnesium stearate; and
(g) about 3.5 wt % to about 15 wt % of a film-coating comprising:
poly(vinyl alcohol);
polyethylene glycol; and
talc.

98. The pharmaceutical dosage form of any one of claims 1 to 91, wherein each granule comprises:
(a) about 33 wt % to about 40 wt % valbenazine ditosylate;
(b) about 20 wt % to about 25 wt % silicified microcrystalline cellulose;
(c) about 16 wt % to about 20 wt % isomalt;
(d) about 6 wt % to about 8 wt % partially pregeletanized maize starch;
(e) about 4 wt % to about 5 wt % hydroxypropyl methylcellulose;
(f) about 2 wt % to about 2.5 wt % magnesium stearate; and
(g) about 8 wt % to about 11 wt % of the film-coating.

99. The pharmaceutical dosage form of any one of claims 1 to 91, wherein each granule comprises:
(a) about 33 wt % to about 40 wt % valbenazine ditosylate;
(b) about 20 wt % to about 25 wt % silicified microcrystalline cellulose;
(c) about 16 wt % to about 20 wt % isomalt;
(d) about 6 wt % to about 8 wt % partially pregeletanized maize starch;
(e) about 4 wt % to about 5 wt % hydroxypropyl methylcellulose;
(f) about 2 wt % to about 2.5 wt % magnesium stearate; and
(e) about 8 wt % to about 11 wt % of the film-coating, wherein the film-coating comprises polyvinyl alcohol, polyethylene glycol, talc, and titanium dioxide.

100. The pharmaceutical dosage form of any one of claims 1 to 91, wherein each granule comprises:
(a) about 33 wt % to about 40 wt % valbenazine ditosylate;
(b) about 20 wt % to about 25 wt % silicified microcrystalline cellulose;
(c) about 16 wt % to about 20 wt % isomalt;
(d) about 6 wt % to about 8 wt % partially pregeletanized maize starch;
(e) about 4 wt % to about 5 wt % hydroxypropyl methylcellulose;
(f) about 2 wt % to about 2.5 wt % magnesium stearate; and
(g) about 8 wt % to about 11 wt % OPADRY® II.

101. The pharmaceutical dosage form of any one of claims 1 to 91, wherein each granule comprises:
(a) about 34.5 wt % to about 38.2 wt % valbenazine ditosylate;
(b) about 22.6 wt % to about 24 wt % silicified microcrystalline cellulose;
(c) about 17.3 wt % to about 19.2 wt % isomalt;
(d) about 6.5 wt % to about 7.2 wt % partially pregeletanized maize starch;
(e) about 4.4 wt % to about 5 wt % hydroxypropyl methylcellulose;
(f) about 2 wt % to about 2.3 wt % magnesium stearate; and
(g) about 8.2 wt % to about 10.5 wt % of the film-coating.

102. The pharmaceutical dosage form of any one of claims 1 to 91, wherein each granule comprises:
(a) about 34.5 wt % to about 38.2 wt % valbenazine ditosylate;
(b) about 22.6 wt % to about 24 wt % silicified microcrystalline cellulose;
(c) about 17.3 wt % to about 19.2 wt % isomalt;
(d) about 6.5 wt % to about 7.2 wt % partially pregeletanized maize starch;
(e) about 4.4 wt % to about 5 wt % hydroxypropyl methylcellulose;
(f) about 2 wt % to about 2.3 wt % magnesium stearate; and
(g) about 8.2 wt % to about 10.5 wt % of the film-coating, wherein the film-coating comprises polyvinyl alcohol, polyethylene glycol, talc, and titanium dioxide.

103. The pharmaceutical dosage form of any one of claims 1 to 91, wherein each granule comprises:
(a) about 34.5 wt % to about 38.2 wt % valbenazine ditosylate;
(b) about 22.6 wt % to about 24 wt % silicified microcrystalline cellulose;
(c) about 17.3 wt % to about 19.2 wt % isomalt;
(d) about 6.5 wt % to about 7.2 wt % partially pregeletanized maize starch;
(e) about 4.4 wt % to about 5 wt % hydroxypropyl methylcellulose;
(f) about 2 wt % to about 2.3 wt % magnesium stearate; and
(g) about 8.2 wt % to about 10.5 wt % OPADRY® II.

104. The pharmaceutical dosage form of any one of claims 1 to 103, wherein each granule comprises an average amount of valbenazine, or a pharmaceutically acceptable salt thereof, of about 1.5 mg to about 2.5 mg, based on the weight of the free base.

105. The pharmaceutical dosage form of any one of claims 1 to 103, wherein each granule comprises an average amount of valbenazine, or a pharmaceutically acceptable salt thereof, of about 1.8 mg to about 2.2 mg, based on the weight of the free base.

106. The pharmaceutical dosage form of any one of claims 1 to 103, wherein each granule comprises an average amount of valbenazine, or a pharmaceutically acceptable salt thereof, of about 1.85 mg to about 2.15 mg, based on the weight of the free base.

107. The pharmaceutical dosage form of any one of claims 1 to 103, wherein each granule comprises an average amount of valbenazine, or a pharmaceutically acceptable salt thereof, of about 1.9 mg to about 2.1 mg, based on the weight of the free base.

108. The pharmaceutical dosage form of any one of claims 1 to 103, wherein each granule comprises an average amount of valbenazine, or a pharmaceutically acceptable salt thereof, of about 1.95 mg to about 2.05 mg, based on the weight of the free base.

109. The pharmaceutical dosage form of any one of claims 1 to 103, wherein each granule comprises an average amount of valbenazine, or a pharmaceutically acceptable salt thereof, of about 2 mg, based on the weight of the free base.

110. The pharmaceutical dosage form of any one of claims 1 to 103, wherein each granule comprises an average amount of valbenazine ditosylate of about 3 mg to about 4.5 mg.

111. The pharmaceutical dosage form of any one of claims 1 to 103, wherein each granule comprises an average amount of valbenazine ditosylate of about 3.4 mg to about 4 mg.

112. The pharmaceutical dosage form of any one of claims 1 to 103, wherein each granule comprises an average amount of valbenazine ditosylate of about 3.5 mg to about 3.9 mg.

113. The pharmaceutical dosage form of any one of claims 1 to 103, wherein each granule comprises an average amount of valbenazine ditosylate of about 3.6 mg to about 3.8 mg.

114. The pharmaceutical dosage form of any one of claims 1 to 103, wherein each granule comprises an average amount of valbenazine ditosylate of about 3.7 mg.

115. The pharmaceutical dosage form of any one of claims 1 to 114, wherein the pharmaceutical dosage form is a capsule.

116. The pharmaceutical dosage form of claim 115, wherein the capsule is a size 00 or smaller.

117. The pharmaceutical dosage form of claim 115, wherein the capsule is a size 00.

118. The pharmaceutical dosage form of claim 115, wherein the capsule is a size 0.

119. The pharmaceutical dosage form of claim 115, wherein the capsule is a size 1.

120. The pharmaceutical dosage form of claim 115, wherein the capsule is a size 2.

121. The pharmaceutical dosage form of any one of claims 115 to 120, wherein the capsule is a sprinkle capsule.

122. A unit dosage form comprising the pharmaceutical dosage form of any one of claims 1 to 121, wherein the valbenazine, or a pharmaceutically acceptable salt thereof, is present in an amount of about 10 mg to about 200 mg, based on the weight of the free base.

123. The unit dosage form of claim 122, wherein the valbenazine, or a pharmaceutically acceptable salt thereof, is present in an amount of about 20 mg to about 80 mg, based on the weight of the free base.

124. The unit dosage form of claim 122, wherein the valbenazine, or a pharmaceutically acceptable salt thereof, is present in an amount of about 10 mg, based on the weight of the free base.

125. The unit dosage form of claim 122, wherein the valbenazine, or a pharmaceutically acceptable salt thereof, is present in an amount of about 20 mg, based on the weight of the free base.

126. The unit dosage form of claim 122, wherein the valbenazine, or a pharmaceutically acceptable salt thereof, is present in an amount of about 40 mg, based on the weight of the free base.

127. The unit dosage form of claim 122, wherein the valbenazine, or a pharmaceutically acceptable salt thereof, is present in an amount of about 60 mg, based on the weight of the free base.

128. The unit dosage form of claim 122, wherein the valbenazine, or a pharmaceutically acceptable salt thereof, is present in an amount of about 80 mg, based on the weight of the free base.

129. The unit dosage form of claim 122, wherein the valbenazine, or a pharmaceutically acceptable salt thereof, is present in an amount of about 100 mg, based on the weight of the free base.

130. A method of administering valbenazine, or a pharmaceutically acceptable salt thereof, to a patient in need thereof, comprising:
(a) providing the pharmaceutical dosage form of any one of claims 1 to 121 or the unit dosage form of claims 122 to 129;
(b) sprinkling the plurality of granules on soft food; and
(c) administering the soft food orally.

131. The method of claim 130, wherein the soft food is chosen from applesauce, yogurt, pudding, ice cream, baby food, and a soy or grain-based product.

132. A method of administering valbenazine, or a pharmaceutically acceptable salt thereof, to a patient in need thereof, comprising: orally administering the pharmaceutical dosage form of any one of claims 1 to 121 or the unit dosage form of claims 122 to 129.

133. A method of administering valbenazine, or a pharmaceutically acceptable salt thereof, to a patient in need thereof, comprising: orally administering a capsule containing the pharmaceutical dosage form of any one of claims 1 to 121 or the unit dosage form of claims 122 to 129.

134. A method of treating a neurological or psychiatric disease or disorder in a patient in need thereof, comprising administering to the patient a therapeutically effective amount of the pharmaceutical dosage form of any one of claims 1 to 121 or the unit dosage form of claims 122 to 129.

135. The method of claim 134, wherein the patient has dysphagia.

136. The method of claim 134 or 135, wherein the patient is a pediatric patient.

137. The method of any one of claims 134 to 136, wherein the neurological or psychiatric disease or disorder is a hyperkinetic movement disorder, mood disorder, bipolar disorder, schizophrenia, schizoaffective disorder, mania in mood disorder, depression in mood disorder, treatment-refractory obsessive compulsive disorder, neurological dysfunction associated with Lesch-Nyhan syndrome, agitation associated with Alzheimer's disease, Fragile X syndrome or Fragile X-associated tremor-ataxia syndrome, autism spectrum disorder, Rett syndrome, or chorea-acanthocytosis.

138. The method of any one of claims 134 to 136 wherein the neurological or psychiatric disease or disorder is a hyperkinetic movement disorder.

139. The method of claim 138, wherein the hyperkinetic movement disorder is tardive dyskinesia.

140. The method of claim 138, wherein the hyperkinetic movement disorder is Tourette's syndrome.

141. The method of claim 138, wherein the hyperkinetic movement disorder is Huntington's disease.

142. The method of claim 138, wherein the hyperkinetic movement disorder is tics.

143. The method of claim 138, wherein the hyperkinetic movement disorder is chorea associated with Huntington's disease.

144. The method of claim 138, wherein the hyperkinetic movement disorder is ataxia, chorea, dystonia, Huntington's disease, myoclonus, restless leg syndrome, or tremors.

145. The method of any one of claims 134 to 144, wherein the patient has 22q11.2 deletion syndrome.

146. The method of any one of claims 134 to 144, wherein the patient is predisposed to developing a psychiatric disorder due to the patient having 22q11.2 deletion syndrome.

147. The method of any one of claims 134 to 144, wherein the patient has COMT haploinsufficiency.

148. The method of any one of claims 134 to 144, wherein the patient is predisposed to developing a psychiatric disorder due to the patient having COMT haploinsufficiency.

149. A process for preparing granules comprising an amount of valbenazine, or a pharmaceutically acceptable salt thereof, the process comprising:
(1) roller compacting a blend comprising milled isomalt (item 3), milled valbenazine ditosylate (item 1), milled silicified microcrystalline cellulose (item 2), milled partially pregelatinized maize starch (item 4), milled hypromellose (item 5), and sieved magnesium stearate (item 6) to obtain roller compacted ribbon material;
(2) milling the roller compacted ribbon material;
(3) blending additional sieved magnesium stearate and the milled roller compacted ribbon material of step (2) to obtain a final blend; and
(4) pressing the final blend to obtain granules.

150. A process for preparing granules comprising an amount of valbenazine, or a pharmaceutically acceptable salt thereof, the process comprising:
(1) blending isomalt (item 3), valbenazine ditosylate (item 1), and silicified microcrystalline cellulose (item 2);
(2) milling the mixture of step (1) and transferring the milled material to a bin;
(3) blending the mixture of step (2);
(4) milling partially pregelatinized maize starch (item 4) and hypromellose (item 5) and adding the milled mixture comprising item 4 and item 5 to the mixture of step (3);
(5) blending the mixture of step (4);
(6) adding sieved magnesium stearate (item 6) to the mixture of step (5);
(7) blending the mixture of step (6);
(8) roller compacting the mixture of step (7) to obtain roller compacted ribbon material;
(9) milling the roller compacted ribbon material;
(10) blending additional sieved magnesium stearate and the milled roller compacted ribbon material of step (9) to obtain a final blend; and
(11) pressing the final blend to obtain granules.

151. The process of claim 149 or 150 further comprising coating the granules with a film-coating.

152. The process of claim 151, wherein the film-coating comprises OPADRY® II.
